(19) United States
(12) Patent Application Publication
(10) Pub. No.: US 2021/0251636 A1
(43) Pub. Date: Aug. 19, 2021

(54) HEMOSTASIS DEVICE WITH CUSTOMIZABLE STRAPS

(71) Applicant: Terumo Medical Corporation, Somerset, NJ (US)

(72) Inventor: Victoria Moore O'Brien, Sewell, NJ (US)

(73) Assignee: Terumo Medical Corporation, Somerset, NJ (US)

(21) Appl. No.: 16/794,813

(22) Filed: Feb. 19, 2020

(51) Int. Cl. A61B 17/132; A61B 17/135

(52) U.S. Cl. CPC A61B 17/1325; A61B 2017/00557; A61B 17/1327; A61B 17/135

(57) ABSTRACT

The present application discloses various embodiments of a hemostasis device in the form of a kit having a compression element and a plurality of adjustable straps that permit for a customized fit of the hemostasis device on varied anatomy.

### BACKGROUND

The present disclosure relates to a closure band adapted to act as a compression device to promote hemostasis at a surgical access site, and more particularly to a vascular closure band with a customizable strap arrangement.

After a surgical procedure involving arterial or venous access, it may be desirable or necessary to apply pressure to the vascular access site to promote hemostasis. Existing closure bands-some of which are in an annular shape—have been used in the past to apply pressure to the access site, regardless of the location of the access site on the body. When used on some portions of the body having highly-tapered or irregular shapes—for example the hand, foot, or obese wrists-such bands may tend to migrate, thus reducing the compressive effectiveness of the device.

Accordingly, there is a need for a vascular closure band that addresses these and other drawbacks of the prior art.

### SUMMARY OF THE DISCLOSURE

In one respect, the present disclosure comprises a hemostasis device comprising: a main portion, the main portion comprising at least one compression element that is adapted to be placed atop a site on a patient where bleeding is to be stopped, and at least one attachment portion, the at least one compression element capable of generating an applied force; a first strap, the first strap being adapted to be wrapped around a body part of the patient and releasably attached to itself or to the at least one attachment portion of the main portion, the first strap having an axial centerline, the first strap being resistant to stretching such that it directs the applied force of the at least one compression element toward the site on the patient when the first strap is wrapped and releasably attached around the body part with the at least one compression element located atop the site on the patient; and a second strap, the second strap being adapted to be wrapped around a body part of the patient and releasably attached to the main portion or the first strap, the second strap having an axial centerline, wherein the second strap may be oriented at at least two different angles such that its axial centerline is not aligned with the axial centerline of the first strap in either of the at least two different angles.

In another respect, the present disclosure comprises a kit for promoting hemostasis at a site on a patient, the kit comprising: a main portion, the main portion comprising at least one compression element and an axial centerline; a first strap, the first strap being removably attachable at both ends thereof to the main portion, the first strap further comprising an axial centerline; and a second strap, the second strap being removably attachable at both ends thereof to either the main portion or the first strap, the second strap further comprising an axial centerline; wherein each of said axial centerlines may be arranged in a non-linear configuration with respect to each other when the kit is being employed on the patient for the promotion of hemostasis at the site.

In yet another respect, the present disclosure comprises a kit for promoting hemostasis at a site on a patient, the kit comprising: a main portion, the main portion comprising at least one compression element and a means for securing a plurality of straps thereto; a first strap of the plurality of straps, the first strap being removably attachable at both ends thereof to the main portion via the means for securing; and a second strap of the plurality of straps, the second strap being removably attachable at both ends thereof to the main portion via the means for securing.

### BRIEF DESCRIPTION OF THE DRAWINGS

The present disclosure will hereinafter be described in conjunction with the appended drawing figures, wherein like numerals denote like elements.

FIG. 1 is a front view of a hemostasis device according to an embodiment of the present disclosure, in the form of a kit comprising a main portion having a compression element and a plurality of straps;

FIG. 2 is a front view of a main portion of a hemostasis device according to another embodiment of the present disclosure;

FIG. 3 is a front view of a main portion of a hemostasis device according to another embodiment of the present disclosure;

FIG. 4 is a front view of a main portion of a hemostasis device according to another embodiment of the present disclosure;

FIG. 5 is a front view of a plurality of straps for a hemostasis device according to the present disclosure;

FIG. 6 is a front perspective view of a strap for a hemostasis device according to another embodiment of the present disclosure;

FIG. 7 is a partial view of the area indicated by 7-7 of FIG. 6;

FIGS. 8 and 9 show the hemostasis device of FIG. 1 attached atop a Dorsalis Pedis arterial access site on a foot, according to an embodiment of a wrapping configuration and method;

FIGS. 10 and 11 show the hemostasis device of FIG. 1 attached atop a Dorsalis Pedis arterial access site on a foot, according to another embodiment of a wrapping configuration and method;

FIG. 12 shows the hemostasis device of FIG. 1 attached atop a Posterior Tibial arterial access site on a foot, according to an embodiment of a wrapping configuration and method;

FIG. 13 shows the hemostasis device of FIG. 1 attached atop a Posterior Tibial arterial access site on a foot, according to another embodiment of a wrapping configuration and method;

FIG. 14 shows the hemostasis device of FIG. 1 attached atop a Posterior Tibial arterial access site on a foot, according to yet another embodiment of a wrapping configuration and method;

FIGS. 15 and 16 show the hemostasis device of FIG. 1 attached atop a Distal Radial arterial access site on a left hand, according to an embodiment of a wrapping configuration and method;

FIG. 17 shows the hemostasis device of FIG. 1 attached atop a Distal Radial arterial access site on a right hand, according to an embodiment of a wrapping configuration and method;

FIG. 18 shows the hemostasis device of FIG. 1 attached atop a Distal Radial arterial access site on a right hand, according to another embodiment of a wrapping configuration and method;

FIG. 19 is a perspective, schematic view of another embodiment of a hemostasis device according to the present disclosure;

FIG. 20 is a perspective, schematic view of another embodiment of a hemostasis device according to the present disclosure;

FIG. 21 is a front view of a hemostasis device according to an alternative embodiment of the present disclosure, in the form of a kit comprising a main portion having a compression element and a plurality of straps;

FIG. 22 is a front view of a hemostasis device according to another alternative embodiment of the present disclosure, in the form of a kit comprising a main portion having a compression element and a plurality of straps;

FIG. 23 is a front view of a hemostasis device according to yet another alternative embodiment of the present disclosure, in the form of a kit comprising a main portion having a compression element and a plurality of straps;

FIG. 24 shows the hemostasis device of FIG. 21 attached atop a Dorsalis Pedis arterial access site on a foot, according to an embodiment of a wrapping configuration and method;

FIG. 25 shows the hemostasis device of FIG. 23 attached atop a Dorsalis Pedis arterial access site on a foot, according to an embodiment of a wrapping configuration and method;

FIG. 26 is a front view of a hemostasis device according to an alternative embodiment of the present disclosure, in the form of a kit comprising a main portion having a compression element and a plurality of straps;

FIG. 27 shows the hemostasis device of FIG. 26 attached atop a Dorsalis Pedis arterial access site on a foot, according to an embodiment of a wrapping configuration and method;

FIG. 28 is a front view of a hemostasis device according to an alternative embodiment of the present disclosure, in the form of a kit comprising a main portion having a compression element and a plurality of straps;

FIG. 29 shows the hemostasis device of FIG. 28 attached atop a Dorsalis Pedis arterial access site on a foot, according to an embodiment of a wrapping configuration and method;

FIG. 30 is a top perspective view of a main portion of a hemostasis device according to the embodiment of FIGS. 28-29;

FIG. 31 is a partial exploded view showing a rotating assembly thereof;

FIG. 32 is a sectional view taken along line 32-32 of FIG. 31;

FIG. 33 is a front view of a hemostasis device according to an alternative embodiment of the present disclosure, in the form of a kit comprising a main portion having a compression element and a plurality of straps;

FIG. 34 shows the hemostasis device of FIG. 33 attached atop a Dorsalis Pedis arterial access site on a foot, according to an embodiment of a wrapping configuration and method;

FIG. 35 is a front view of a hemostasis device according to an alternative embodiment of the present disclosure, in the form of a kit comprising a main portion having a compression element and a plurality of straps;

FIG. 36 is a front view of a hemostasis device according to an alternative embodiment of the present disclosure, in the form of a kit comprising a main portion having a compression element and a plurality of straps;

FIG. 37 is a front view of a hemostasis device according to an alternative embodiment of the present disclosure, in the form of a kit comprising a main portion having a compression element and a plurality of straps;

FIG. 38 is a front view of a hemostasis device according to an alternative embodiment of the present disclosure, in the form of a kit comprising a main portion having a compression element and a plurality of straps;

FIG. 39 shows the hemostasis device of FIG. 38 attached atop a Distal Radial arterial access site on a left hand, according to an embodiment of a wrapping configuration and method;

FIG. 40 is a schematic, side profile view of an embodiment of a support plate for a main portion of a hemostasis device, according to the present disclosure;

FIG. 41 is a schematic, side profile view of another embodiment of a support plate for a main portion of a hemostasis device, according to the present disclosure;

FIG. 42 is a schematic, side profile view of yet another embodiment of a support plate for a main portion of a hemostasis device, according to the present disclosure;

FIG. 43 is a schematic, side profile view of an embodiment of a semi-rigid support piece for a main portion of a hemostasis device, according to the present disclosure;

FIG. 44 is a schematic, side profile view of an embodiment of a balloon assembly for a main portion of a hemostasis device, according to the present disclosure;

FIG. 45 is a schematic, side profile view of another embodiment of a balloon assembly for a main portion of a hemostasis device, according to the present disclosure;

FIG. 46 is a schematic, side profile view of yet another embodiment of a balloon assembly for a main portion of a hemostasis device, according to the present disclosure;

FIG. 47 is a schematic, side profile view of still another embodiment of a balloon assembly for a main portion of a hemostasis device, according to the present disclosure;

FIG. 48 is a schematic, side profile view of an embodiment of a main portion of a hemostasis device, according to the present disclosure;

FIG. 49 is a schematic, side profile view of another embodiment of a main portion of a hemostasis device, according to the present disclosure; and

FIG. 50 is a schematic, side profile view of yet another embodiment of a main portion of a hemostasis device, according to the present disclosure.

### DETAILED DESCRIPTION OF THE EMBODIMENT(S)

The ensuing detailed description provides exemplary embodiment(s) only, and is not intended to limit the scope, applicability, or configuration thereof. Rather, the ensuing detailed description of the exemplary embodiment(s) will provide those skilled in the art with an enabling description for implementing these embodiment(s). It should be understood that various changes may be made in the function and arrangement of elements of the embodiment(s) without departing from the spirit and scope of the invention, as set forth in the appended claims.

Directional terms (e.g., upper, lower, left, right, etc.) may be used herein. These directional terms are merely intended to assist in disclosing the embodiment(s) and claiming the invention and are not intended to limit the claimed invention in any way. In addition, reference numerals that are introduced in the specification in association with a drawing figure may be repeated in one or more subsequent figure(s) without additional description in the specification, in order to provide context for other features.

Hemostatic devices that are wrapped around a patient's limb at a site on the limb where bleeding is to be stopped, and which include one or more inflatable balloons or bladders that function as compression elements to target pressure at a vascular arterial access site, are known in the art. Multiple embodiments of one such hemostatic device and methods of using such devices are described in U.S. Pat. No. 7,498,477, the entirety of which is incorporated by reference as if set forth herein.

In order for a hemostasis device to properly function, the main or “belt” portion of the hemostasis band needs to do two things: (1) stay in its desired position with the pressure-supplying portion (e.g., a balloon assembly) atop the surgical access site; and (2) direct pressure toward the artery or vein to create hemostasis. The main or belt portion of the hemostasis device (e.g., via a “first strap” of each embodiment described herein) typically acts to direct hemostatic pressure to the vascular access site via hoop stress. This main portion of the hemostasis device resists stretching (via one or more semi-rigid layer(s) or rigid plate), so that when the one or more balloon(s) are inflated, the pressure created by the inflated balloon(s) is directed toward the vascular access site (e.g., an artery or vein) and doesn't cause the main portion to stretch away from the access site. However, even on typical anatomy and without patient movement, a normal force (with respect to the access site) will tend to force a hemostasis device to slide down a tapered surface (e.g., a forearm/wrist, ankle, or foot). Wrapping the band tighter may help to keep the hemostasis device in place on the anatomy, but will cause the edges of the band to dig into the patient's skin, causing discomfort. Wrapping the band in a conical shape (i.e., such that the axial centerlines of the two strap halves are not aligned) may also help prevent such migration, but this configuration may cause misplacement of the balloon and/or a weak connection of the strap due to poor overlap of or connection between the fastener halves, thus increasing the likelihood that the main portion of the hemostasis device will come undone. With reduced fastener overlap, there may be a length of strap not used (i.e., extending past the fastener connection), which may contact the patient's anatomy and cause discomfort. While this excess material could be trimmed or taped down, this creates an extra step for the clinician and is therefore undesirable. Finally, when a hemostasis device is wrapped in such an angled manner, the compression balloon(s) will sit at a different angle with respect to the vascular access site, and thus may not direct the hemostatic force to the vascular access site in the intended manner. Since the force is angled with respect to the vascular access site, some component of the inflation force will tend to push the band out of position, down the taper of the irregular anatomy (e.g., towards the thinner portion of a tapered, obese wrist).

For these reasons, a configurable multi-strap hemostasis band is desirable. The band can be made to fit various anatomies and patient sizes by permitting the user to customize the fit of each strap as appropriate to attain a secure fit. In some embodiments, separate straps can be used, with each strap placed at an appropriate location, tightness, and angle for a secure fit on the patient. In some embodiments according to the present disclosure, a first strap can be used primarily to provide the necessary hoop stress, with a second strap (and possibly additional straps) used to hold the hemostatic device in place such that the main portion thereof (which includes the pressure-supplying component, e.g., one or more inflatable balloons) remains atop the vascular access site in an appropriate orientation with respect to the anatomy. In alternative embodiments according to the present disclosure, multiple separate straps can be used with each strap placed at an appropriate location, tightness, and angle such that more than one strap provides a portion (vector) of the necessary hoop stress, with one or more additional straps optionally included that are used to hold the hemostatic device in place, but that do not provide a significant (or any) vector component of the necessary hoop stress.

In some embodiments according to the present disclosure, one or more of these straps could be flat (i.e., planar), but in alternative embodiments the one or more straps could also be contoured (i.e., non-planar, for example curled or rolled) and/or could have non-linear (i.e., interrupted, for example with “scalloped-shaped”) edges. Various embodiments of hemostasis devices having straps with rolled, curled, scalloped, or otherwise contoured and/or non-linear-edge straps are taught in U.S. patent application Ser. No. 16/288,303, filed Feb. 28, 2019, and U.S. patent application Ser. No. 29/682,051, filed Mar. 1, 2019, the entire contents of which are incorporated herein by reference as if set forth in their entireties.

The multiple straps provided on such a hemostasis device can tangle if they are long and already attached to the main portion thereof (i.e., the portion including the compression balloon(s)). Separable straps are easier for a clinician to manage. Separate straps can be attached at any angle, permitting additional customization for different-sized people, relative sizes of different anatomical areas, and/or tapered- or irregularly-shaped anatomical geometries. Infinitely-adjustable fasteners, such as hook and loop or some styles of belt fasteners, also permit for the length of each strap to be precisely customized. In some embodiments according to the present disclosure, two-sided hook and loop fastener is used, although other types of fastener are suitable, as would be appreciated by a person having ordinary skill in the art. For example, in some embodiments, self-adhering fastener material could be used that comprises both hook and loop material contained within the same fastener face. In some embodiments, use of such self-adhering fastener material would permit for greater length adjustability as compared to the use of separate halves of different material (e.g., separate fastener halves comprising, respectively, hook material and loop material).

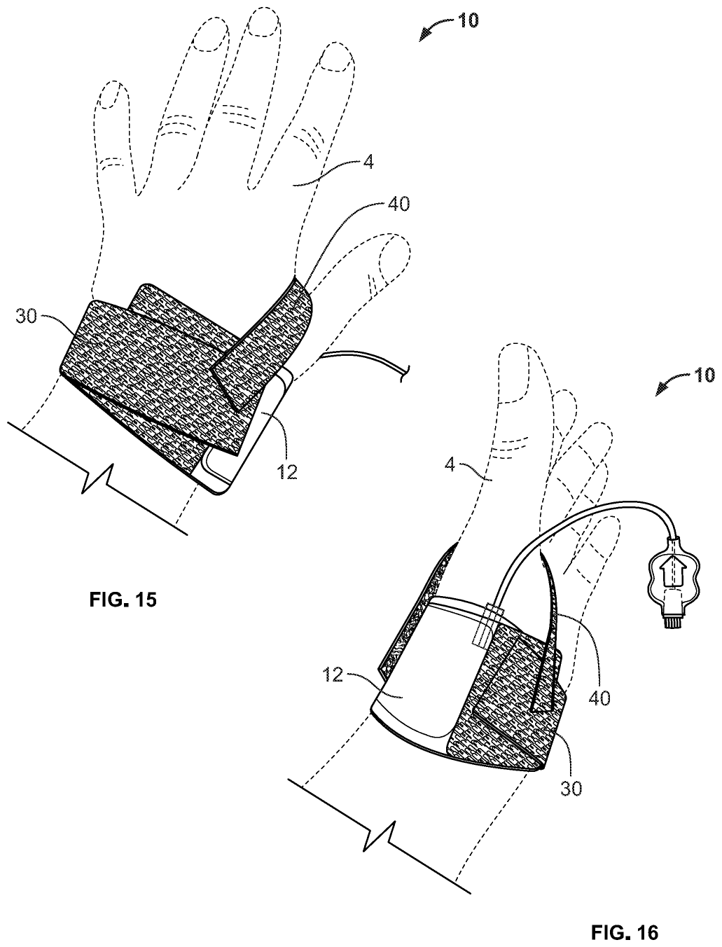

An important feature of the hemostasis devices according to the present disclosure is the ability to adjust straps at any angle and locate them in multiple positions, to fit various anatomical locations and patient sizes. Although elasticized pieces may stay in place when flexed—and tend to be comfortable—the band must resist stretching away from the access site enough to provide equal and opposite hemostatic force against the access site. Portions of the hemostasis device that are only intended to hold the device in place on the patient, and that are not intended to provide the hoop strength necessary to support the hemostatic pressure against the access site (e.g., the second straps according to the various embodiments shown and described herein), may be elasticized. For example, in embodiments according to the present disclosure that are mountable on a patient's hand (see FIGS. 15-18), the strap that goes between the fingers and/or thumb could be elasticized.

In the various embodiments described herein, at least the main portions and first straps (i.e., hoop-stress-providing strap) of the hemostasis devices are transparent and comprised of polyvinyl chloride (PVC), and the second straps are also optionally transparent and comprised of PVC. In alternative embodiments according to the present disclosure, all or portions of the main portion and the strap(s) could be formed from opaque materials and/or alternative types of materials. In further alternative embodiments, the main portion could also optionally be equipped with a patency monitoring sensor. The main portion and strap(s) could also optionally be wipeable in case any of these parts becomes soiled during a procedure.

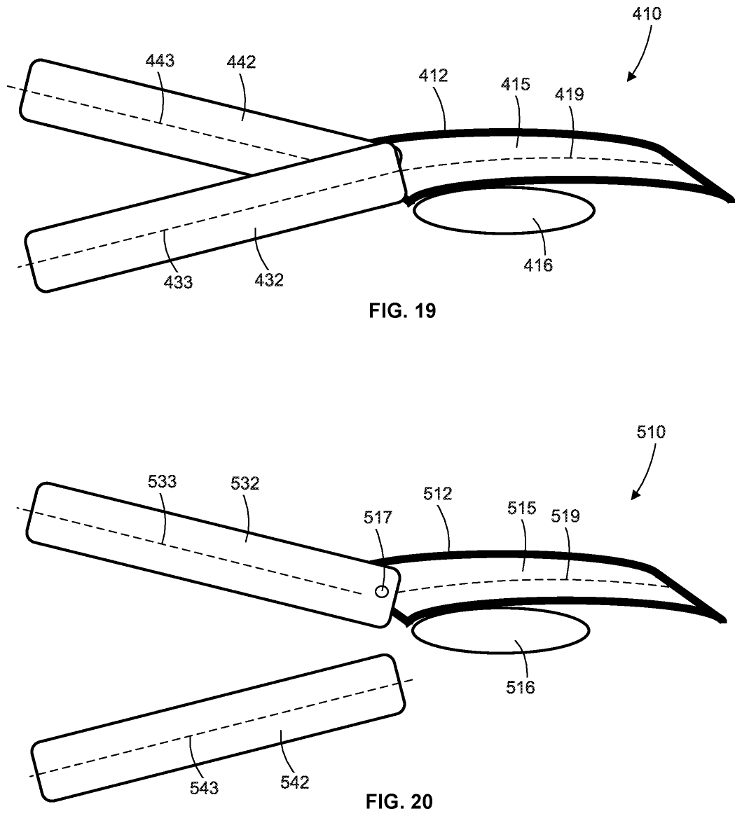

FIGS. 19 and 20 schematically depict two embodiments of a multi-strap hemostasis device (band) 410,510 in accordance with the present disclosure. In each of these embodiments, a respective port on the balloon, an inflator, and an air line that connects between the inflator and the respective port of the balloon have been omitted from view for ease of illustration. In the embodiment of FIG. 19, the hemostasis device 410 comprises a main portion 412 that comprises a rigid plate or semi-rigid layer 415 and at least one inflatable compression element in the form of a balloon 416. The main portion 412 also has an axial centerline 419. The main portion 412 also comprises a first strap 432 having an axial centerline 433 and a second strap 442 having an axial centerline 433. The balloon(s) 416 is placed between the vascular access site and the rigid plate or semi-rigid layer 415, and the rigid plate or semi-rigid layer 415 limits expansion of the balloon 416 outwardly (i.e., away from the patient's body), thus directing the pressure generated by the balloon 416 towards the patient's anatomy. In this embodiment, the straps 432,442 are not separable from the main portion 412 (i.e., are permanently attached thereto), but are each free to be re-oriented (i.e., rotated) such that the angle between their respective axial centerlines 433,443—as well as the angle between either axial centerline 433,443 and the axial centerline 419 of the main portion 412—is infinitely adjustable.

In the embodiment of FIG. 20, the hemostasis device 510 comprises a main portion 512 that comprises a rigid plate or semi-rigid layer 415 and at least one inflatable compression element in the form of a balloon 516. The main portion 512 also has an axial centerline 519. The main portion 512 also comprises a first strap 532 having an axial centerline 533 and a second strap 542 having an axial centerline 533. The balloon(s) 516 is placed between the vascular access site and the rigid plate or semi-rigid layer 515, and the rigid plate or semi-rigid layer 515 limits expansion of the balloon 516 outwardly (i.e., away from the patient's body), thus directing the pressure generated by the balloon 516 towards the patient's anatomy. In this embodiment, the strap 532 is not removable (i.e. detachable) from the main portion 512, but is free to be pivoted about an attachment point with the main portion 512, while the strap 542 is removable from the main portion (as shown in FIG. 20). In this way, each of the straps 532,542 is free to be re-oriented such that the angle between their respective axial centerlines 533,543—as well as the angle between either axial centerline 533,543 and the axial centerline 519 of the main portion 512—is infinitely adjustable.

It should be understood that any of the embodiments of a hemostasis device according to the present disclosure could be modified such that any or all of the straps are pivotable and/or entirely separable from the respective main portion thereof. Providing the multiple straps as detachable and/or angle-able (i.e., pivotable) provides two benefits that are an improvement over the prior art devices: first, it permits the band to be properly angled to provide tension to direct the balloon pressure properly against the target anatomy; and second, it allows the band to be held in place against tapered and/or uneven anatomy. When multiple straps are already attached to a hemostasis device, there is a tendency for those straps to become entangled, rendering the device more difficult to use. Providing a hemostasis device with separable strap pieces permits the straps to be angled as needed to suit different anatomical areas, as well as different sized and shaped patients. Providing a hemostasis device with separable strap pieces would also permit the device to be provided as a kit comprising a variety of straps of different widths, lengths, thicknesses, curvatures, and/or adjustability, which would enable its use on a variety of anatomical features of a large range of patients. While embodiments according to the present disclosure are depicted such that the axial centerline(s) of the one or more strap(s) are shown aligned with the axial centerline of the respective main portion of the hemostasis device, it should be further understood that any of the embodiments of a hemostasis device according to the present disclosure could be configured such that the axial centerline(s) of the one or more strap(s) thereof are not aligned with (i.e., could be spatially offset from) the axial centerline of the respective main portion of the hemostasis device. In various embodiments this non-aligned configuration may be either one of multiple configuration(s) available for the one or more strap (e.g., in embodiments where the strap is removable from the remainder of the hemostasis device), or may be a permanent feature of the one or more strap (e.g., in embodiments where the strap is not removable from the remainder of the hemostasis device, but is instead permanently installed in an offset configuration in relation thereto).

Such a highly-configurable hemostasis device is suitable in multiple clinical scenarios, for example: in situations where physicians might currently modify products off-label; for clinical trials for testing of new surgical or hemostasis procedures; and for hemostasis after rarely-performed interventional procedures, such as where the surgical access is to the left- or right-distal radial, brachial, tibial, or pedal arteries. Custom fits and sizing also provide the benefits of being useable in special contexts, for example for petite, obese, amputated, deformed, variant, or otherwise atypical anatomies.

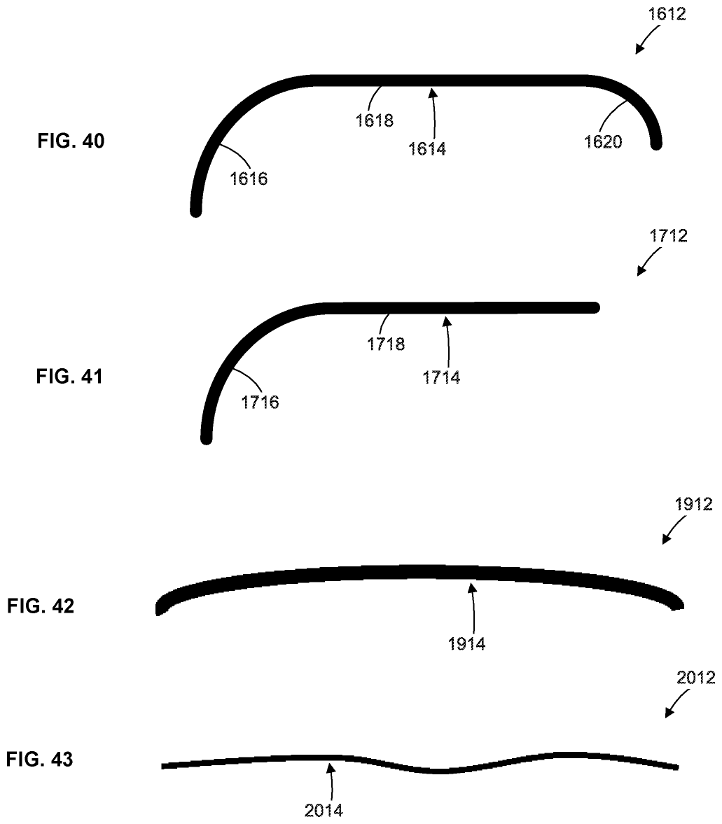

The various multi-strap hemostasis bands according to the present disclosure can include rigid plate(s) or semi-rigid layer(s) of different shapes, sizes, and/or curvatures for different applications, and it should be understood that any of the embodiments taught herein could be modified to include a rigid plate(s) and/or semi-rigid, non-compliant layer(s). As discussed elsewhere herein, these rigid plates or semi-rigid layers direct the balloon pressure towards the patient's anatomy. FIGS. 40-43 show profile views of various plates and semi-rigid layers that may be used with various embodiments in accordance with the present disclosure. FIG. 40 shows a profile view of a support plate 1612 comprising a skin-facing side 1614 and a straight portion 1618 located between a curved portion 1616 and a curved portion 1620. In this embodiment, the curved portions 1616,1620 of the support plate 1612 are used to align the support plate 1612 to specific anatomy (e.g., the radial artery in the wrist/forearm) and protect adjacent anatomy (e.g., the ulnar artery and nerve). FIG. 41 shows a profile view of a support plate 1712 comprising a skin-facing side 1714, a curved portion 1716, and a straight portion 1718. Some plate designs, such as the slightly-curved embodiment of a support plate 1912 having skin-facing side 1914 shown in profile in FIG. 42, can limit expansion of the compression balloon outwardly without features which help align the support plate 1912 to specific anatomical features. Finally, flexible materials, such as the support piece 2012 having skin-facing side 2014 shown in profile in FIG. 43, can also limit expansion of a compression balloon outwardly by having one or more non-compliant material layers. Embodiments of a hemostasis device in accordance with the present disclosure that include these flexible materials are not limited to any specific anatomy, and are thus versatile, but have the downside of not being optimized for any specific anatomy, with the drawbacks of having no alignment shapes, no feature to protect adjacent anatomy, and no shape to improve vector direction of the compressive force generated by the inflated balloon.

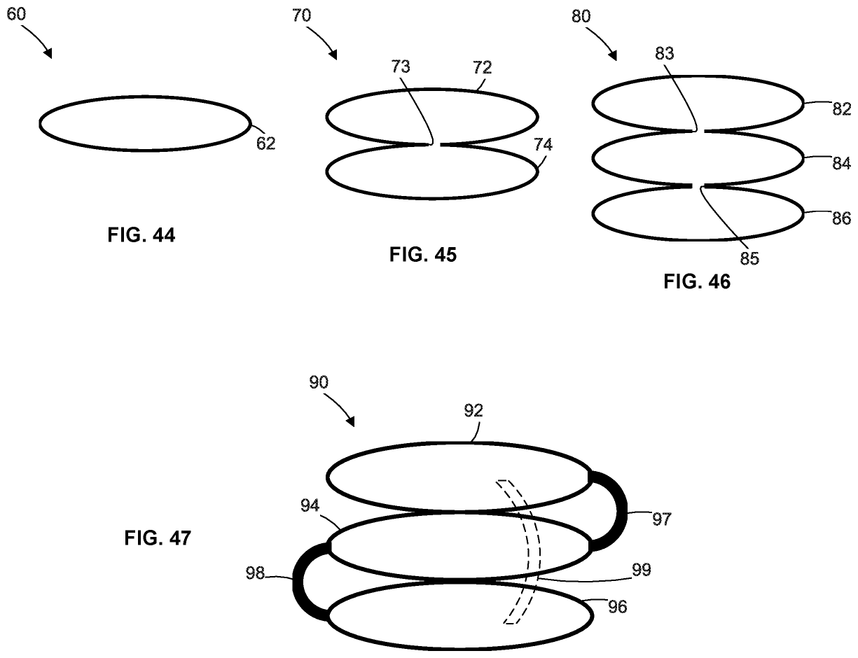

Each of the hemostasis devices according to the embodiments described in the present disclosure also include a balloon assembly comprised of one or more balloons adhered to the main portion of the band (e.g., either to the respective first strap if integral with the rigid plate and/or semi-rigid layer, or else directly to the rigid plate and/or semi-rigid layer). These balloon assemblies can contain one or more separate balloons attached together and/or to the rigid plate or semi-rigid layer in various configurations, as shown schematically in FIGS. 44-50 (with air inlet ports omitted from view). These balloon assemblies can be designed to apply pressure to the vascular access site in either a centered or directed force direction, based on the configuration of the balloons and the location of their attachment point(s) to the main portion of the hemostasis device.

FIG. 44 shows a balloon assembly 60 comprising a single balloon 62. FIG. 45 shows a balloon assembly 70 comprising two balloons 72,74 which are attached together in fluid flow communication via an opening 73 such that the balloons 72,74 are inflatable together to the same internal pressure. FIG. 46 shows a balloon assembly 80 comprising three balloons 82,84,86, with balloons 82,84 connected together in fluid flow communication by an opening 83 and balloons 84,86 connected together in fluid flow communication by an opening 85, such that the balloons 82,84,86 are inflatable together to the same internal pressure. The balloon assemblies 70,80 of FIGS. 45 and 46 could alternatively have the internal openings 73,83,85 omitted and the individual balloons 72,74,82,84,86 could be inflatable via individual air tubes that feed each respective balloon 72,74,82,84,86, either selectively or simultaneously.

Suitable balloon assemblies could also be formed as single units having multiple air chambers which are connected together by one or more air channels, passages, or tubes, and which are inflated together to the same internal pressure, for example balloon assembly 90 of FIG. 47 in which balloon 92 is connected in fluid flow communication with balloon 94 via an air line 97 and balloon 94 is also connected in fluid flow communication with balloon 96 via an air line 98 (and balloons 92,96 are optionally additionally connected together in direct fluid flow communication via air line 99). Suitable balloon assemblies may also comprise various folded balloon designs. Various embodiments of folded balloons designs for a hemostasis device are taught in U.S. Patent Application No. 62/812,436, filed Mar. 1, 2019, the contents of which are incorporated herein by reference as if set forth in its entirety.

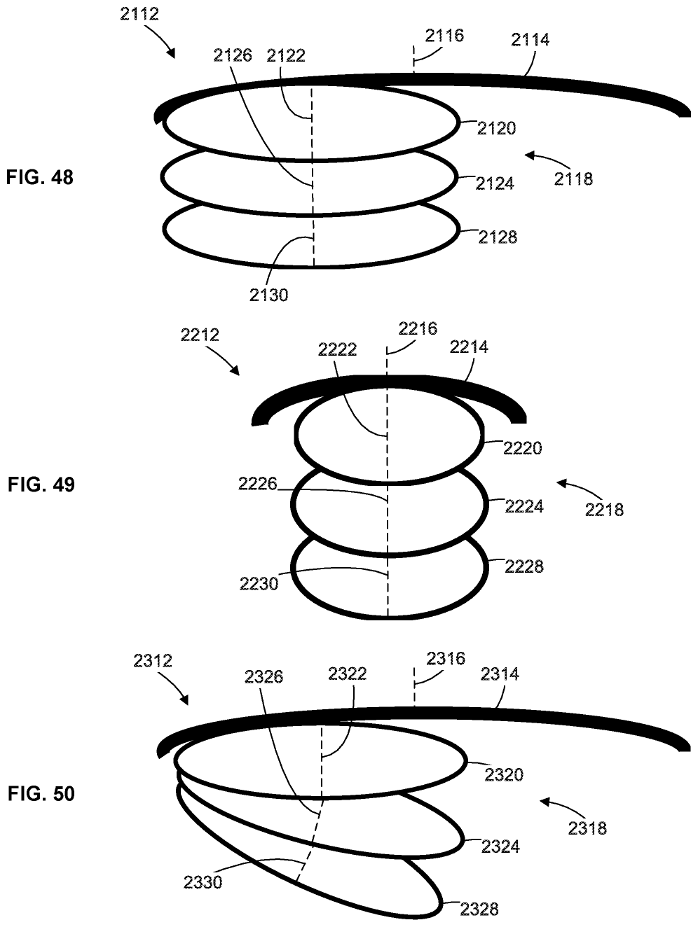

The one or more compression balloon(s) may also be attached (and constrained) to the band, rigid plate(s), or semi-rigid layer(s) in different positions and/or orientations. FIGS. 48-50 show various schematic representations of inflated multi-balloon compression balloon assemblies attached to a respective rigid plate. In the embodiment of FIG. 48, balloon 2120 is attached directly to the underside of support plate 2114 offset from a vertical centerline 2116 of the support plate 2114, balloon 2124 is attached to the underside of balloon 2120, and balloon 2128 is attached to the underside of balloon 2124. The balloons 2120,2124,2128 of the balloon assembly 2118 of this embodiment are attached together in a “stacked” or “column” configuration such that their respective vertical centerlines 2122,2126,2130 are aligned when the balloons 2120,2124,2128 are inflated, but offset from the vertical centerline 2116 of the support plate 2114. The balloon assembly 2118 of this embodiment provides hemostatic force to the access site that is centered with respect to the balloon assembly 2118.

In the embodiment of FIG. 49, balloon 2220 is attached directly to the underside of support plate 2214 aligned with a vertical centerline 2216 of the support plate 2214, balloon 2224 is attached to the underside of balloon 2220, and balloon 2228 is attached to the underside of balloon 2224. The balloons 2220,2224,2228 of the balloon assembly 2218 of this embodiment are attached together in a “stacked” or “column” configuration such that their respective vertical centerlines 2222,2226,2230 are aligned when the balloons 2220,2224,2228 are inflated and also aligned with the vertical centerline 2216 of the support plate 2214. The balloon assembly 2218 of this embodiment provides hemostatic force to the access site that is centered with respect to the balloon assembly 2218.

In the embodiment of FIG. 50, balloon 2320 is attached directly to the underside of support plate 2314 offset from a vertical centerline 2316 of the support plate 2314, balloon 2324 is attached to the underside of balloon 2320, and balloon 2328 is attached to the underside of balloon 2324. The balloons 2320,2324,2328 of the balloon assembly 2318 of this embodiment are attached to each other about a respective edge thereof, with the uppermost balloon 2320 attached to an underside of the support plate 2314, so that when the balloons 2320,2324,2328 are inflated, they create a “fanned” arrangement due to their attachment together about respective side edges, with the respective vertical centerlines 2322,2326,2330 of each of the balloons 2320,2324,2328 being in a non-aligned relationship and also offset from the vertical centerline 2316 of the support plate 2314. The arrangement of the balloons 2320,2324,2328 in the balloon assembly 2318 of the embodiment of FIG. 50 delivers the compressive force thereof at a more oblique or directed angle than the embodiments of the balloon assemblies 2118,2218 shown in FIGS. 48 and 49, in which the compressive force of the balloons is directed in a more linear or centered direction parallel to the vertical centerlines 2122,2126,2130,2222,2226,2230 of the respective balloons 2120,2124,2128,2220,2224,2228. In an alternative embodiment, the balloon assembly 2318 of FIG. 50 could be modified such that the vertical centerline 2322 of the uppermost balloon 2320 in the balloon assembly 2318 is aligned with the vertical centerline 2316 of the support plate 2314.

In embodiments according to the present disclosure, the balloon(s) are partially, substantially, or completely transparent, in order to permit visibility of the vascular access site, both to permit initial placement and to permit monitoring of the site for complications. In embodiments according to the present disclosure, a marker (for example a colored dot or circle) could optionally be located on the balloon(s) to indicate where the balloon(s) should be aligned with respect to the vascular access site.

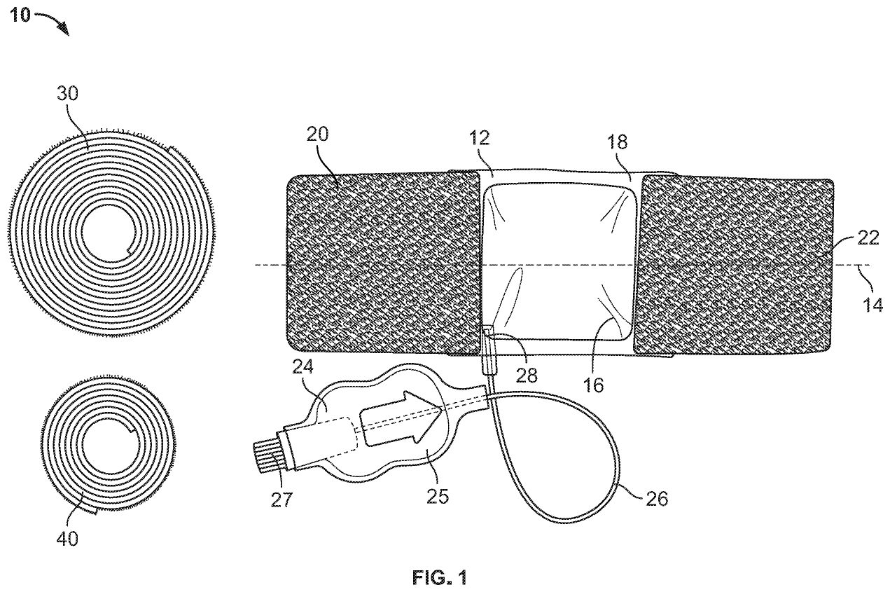

FIG. 1 shows an embodiment of a hemostasis device 10 which is provided as a kit, comprising a main portion 12 that includes a pressure-supplying portion or component, a first strap 30, and a second strap 40. The main portion 12 comprises two attachment portions 20,22, which in this embodiment each comprise a fastener half made of hook-and-loop material. In this embodiment the main portion 12 also comprises an axial centerline 14, a balloon 16, and a non-compliant layer 18 that is comprised of a material that is more rigid than the material from which the balloon 16 is comprised, such that the main portion 12 of the band 10 resists stretching so that when the one or more balloon(s) are inflated the balloon pressure is directed toward the vascular access site and the inflated balloon is prevented from stretching away from the access site.

In this embodiment the hemostasis device 10 further includes an inflator 24 that is used to inflate the balloon 16. An air tube 26 enters the interior of the balloon 16 via a port 28, and the air tube 26 is connected at its opposite end to an inflator 24. In this embodiment, the inflator 24 includes a bulb 25 and a valve housing 27. Inflation of the balloons 16 is achieved by inserting the protruding tip of a syringe (not shown) into the valve housing 27 and pushing a plunger on the syringe so as to introduce fluid (e.g., air) within the syringe through the inflator 24 into the balloon 16. Once fluid has been injected into the balloon 16 and the protruding tip of the syringe is withdrawn from the valve housing 27, a check valve (not shown) within the valve housing 27 closes, preventing the fluid from leaking out and thus maintaining the balloon 16 in an inflated state. It would be understood by one of ordinary skill in the art that any suitable known or hereafter-disclosed inflation mechanisms or methods may be used with any of the hemostasis devices according to the present disclosure, though a detailed discussion of such alternatives is outside the scope of the present disclosure.

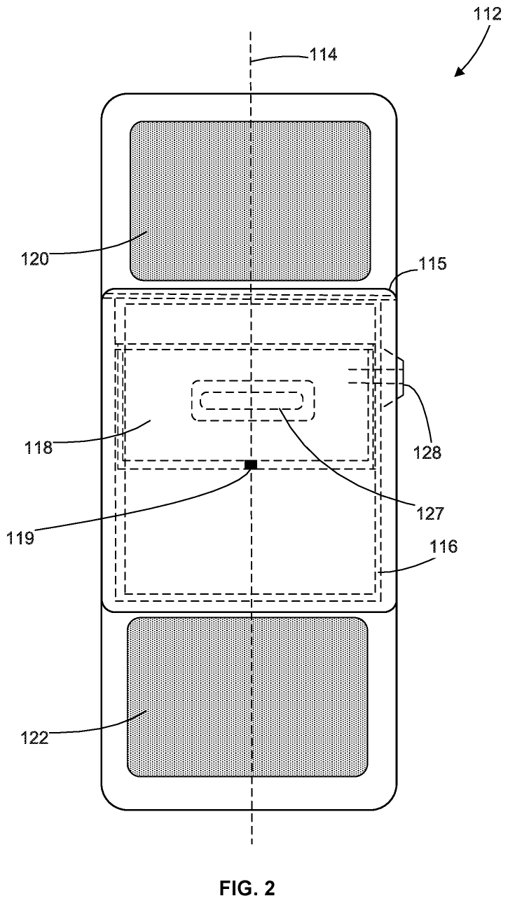

FIGS. 2-4 show additional, alternative embodiments of a main portion of a hemostasis device in accordance with the present disclosure. In each of these embodiments, an inflator and an air line that connects between the inflator and the respective port of the balloon assembly have been omitted from view for ease of illustration. FIG. 2 shows another embodiment of a main portion 112 of a hemostasis device according to another embodiment of the present disclosure. In this embodiment, the main portion 112 comprises an axial centerline 114, a main balloon 116, and a secondary balloon 118. An opening 127 exists between an interior of the main balloon 116 and an interior of the secondary balloon 118, so that the secondary balloon 118 inflates as the main balloon 116 inflates, providing for efficient ease of use. Air is introduced into the main balloon 116 via a port 128 through known means as discussed elsewhere herein and as would be understood by those of ordinary skill in the art. When both balloons 116,118 are inflated, the secondary balloon 118 provides oblique pressure against the main balloon 116, which in turn provides directed pressure to the respective artery or vein, thus promoting hemostasis. In this embodiment, the main portion 112 further includes a marker 119 located on an interior edge of the secondary balloon 118 (and approximately in the center of the main balloon 116), which assists the clinician in aligning the balloons 116,118 over the center of the vascular access site. In alternative embodiments according to the present disclosure, the marker 119 could be located directly on the main balloon 116 in approximately its center. In further alternative embodiments according to the present disclosure, the marker 119 or other alignment indicator(s) could be located elsewhere on the balloon assembly or hemostasis device, or could be omitted entirely. It should further be understood that any of the embodiments of the main portion 12,112,212,312 taught herein could employ any balloon assembly comprising one or more balloons or inflatable portions, as provided elsewhere in the present disclosure, without departing from the scope or spirit hereof.

In the embodiment of FIG. 2, the main portion 112 is further provided with a rigid plate 115 located above the balloons 116,118 which acts to direct the balloon pressure towards the access site. It should be understood that the rigid plate 115 is an optional feature of the embodiment of FIG. 2, and that this embodiment could alternatively be provided with a balloon having a non-compliant top layer, as in the embodiment according to FIG. 1. It should be further understood that any of the embodiments shown and described in the present disclosure could be provided with either or both of a balloon assembly having a non-compliant upper layer or rigid plate, without departing from the scope of the present disclosure.

The embodiment of FIG. 2 further comprises a first attachment portion 120 having a fastener half (e.g., hook-and-loop fastener patch) thereon, and a second attachment portion 122 having a fastener half (e.g., hook-and-loop fastener patch) thereon. Unlike the embodiment of FIG. 1, in the embodiment of FIG. 2 the fastener patches do not cover the entire surfaces of the main portion 112 that extend from the balloons 116,118, but like the embodiment of FIG. 1, the embodiment of FIG. 2 includes two attachment portions 120,122 that are located on opposite sides of the balloons 116,118.

FIG. 3 shows another embodiment of a main portion 212 of a hemostasis device according to another embodiment of the present disclosure. In this embodiment, the main portion 212 comprises an axial centerline 214, a main balloon 216 and a secondary balloon 218 attached in fluid flow communication by an opening 227 and fed by a port 228, a marker 219 located on an interior edge of the secondary balloon 218, and a rigid plate 215 optionally located above the balloons 216,218. The embodiment of FIG. 3 further comprises a single attachment portion 220 including a fastener half (e.g., hook-and-loop fastener patch) thereon, with the attachment portion 220 entirely surrounding the perimeter of the balloons 216,218 in a “square” configuration and forming the entire outer perimeter of the main portion 212.

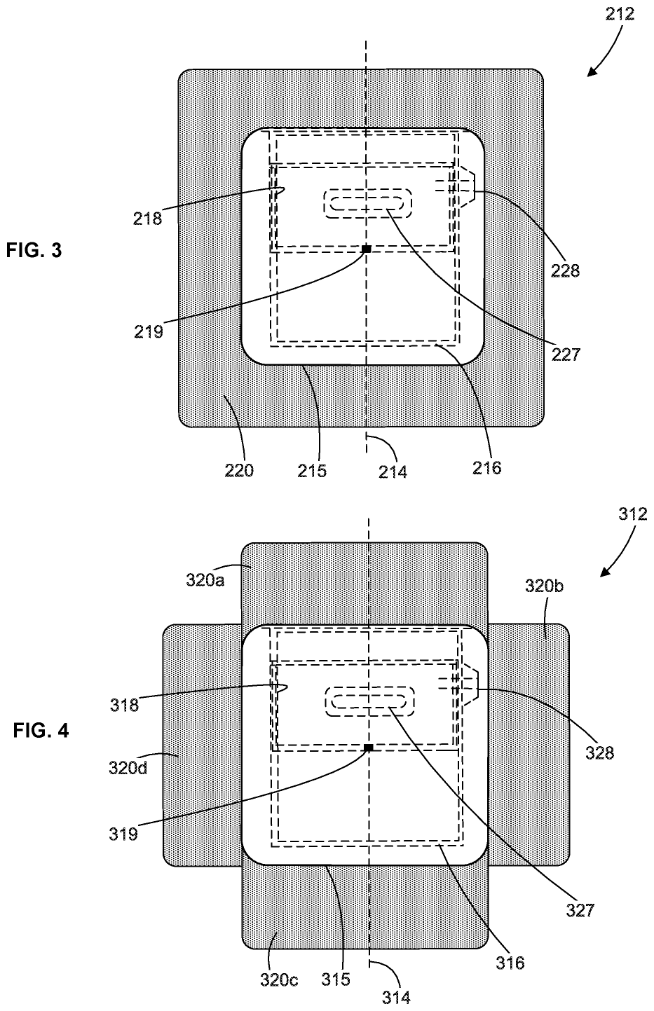

FIG. 4 shows yet another embodiment of a main portion 312 of a hemostasis device according to an alternative embodiment of the present disclosure. In this embodiment, the main portion 312 comprises an axial centerline 314, a main balloon 316 and a secondary balloon 318 attached in fluid flow communication by an opening 327 and fed by a port 328, a marker 319 located on an interior edge of the secondary balloon 318, and a rigid plate 315 optionally located above the balloons 316,318. The embodiment of FIG. 4 further comprises four attachment portions 320a-320d, each including a fastener half (e.g., hook-and-loop fastener patch) thereon, with the attachment portions 320a-320d arranged on all four sides of the balloons 316,318 in a “plus”-shaped configuration.

Each of the arrangements of the main portions 12,112,212,312 show in FIGS. 1-4 are suitable for various hemostasis applications, and any number of them could be included along with a plurality of straps to form various special-purpose or general-purpose hemostasis kits, in accordance with the present disclosure. Within the scope of the present disclosure, clinicians of ordinary skill in the art will be able to employ the main portions 12,112,212,312 and straps taught herein in any number of various configurations for proper alignment and directing of force towards a surgical access site after both currently-known and new types of surgical procedures.

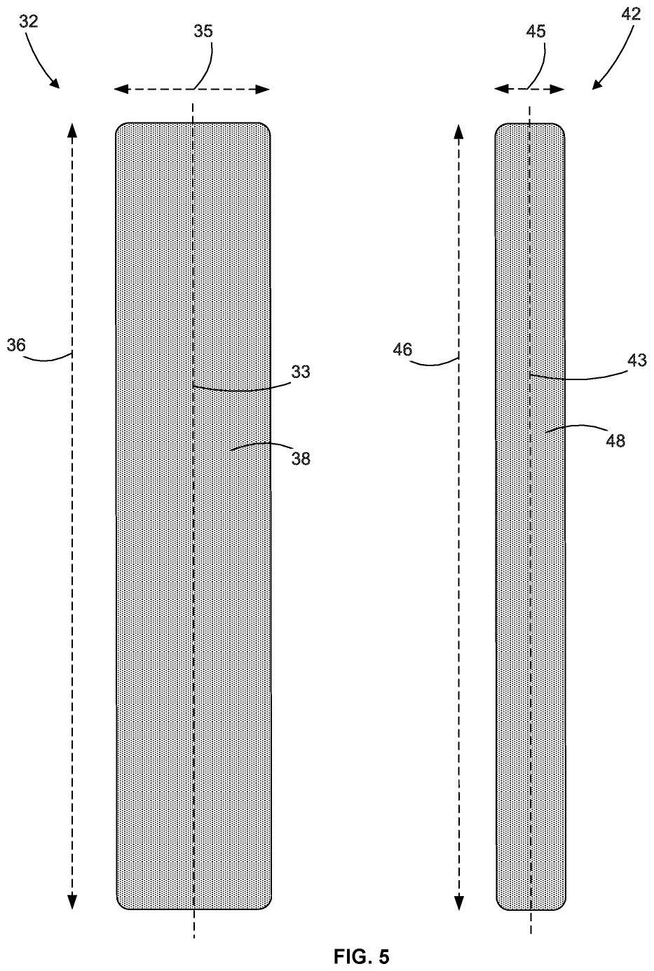

FIG. 5 shows two straps 32,42 according to any embodiment of a hemostasis device according to the present disclosure. In this embodiment, each of the straps 32,42 is flat (planar), of fixed width 35,45 and length 45,46, and entirely covered on one or both sides with an attachment portion 38,48 (e.g., a fastener half such as hook-and-loop or both hook-and-loop on the same face). Strap 32 has an axial centerline 33 and strap 42 has an axial centerline 43. In alternative embodiments, either or both of the straps 32,42 could be of variable or custom-selectable actual length, “effective length” (i.e., the length of the strap as measured between its attachment point with the main portion of the hemostasis device and its attachment point distal thereto that is used to secure the strap back to the main portion after it has been wrapped around some portion of the patient's anatomy), and/or width, could have a non-planar surface as discussed elsewhere herein, and/or could have one or more attachment portions that do not cover the entirety of a side thereof. Moreover, any hemostasis device according to the present disclosure could be provided with any number of strap pieces as part of a kit provided to a clinician, or could be provided along with long roll(s) of strap that could be cut to size by the clinician according to the specific hemostasis application, either via a separate cutting tool or a built-in strap cutter provided as part of the hemostasis kit.

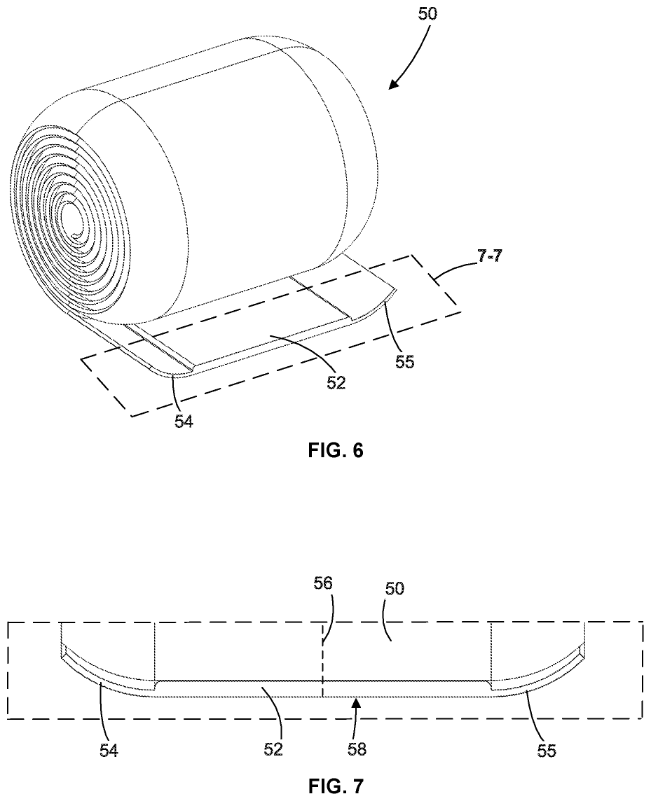

FIG. 6 shows a roll of strap 50 according to another embodiment of the present disclosure, and FIG. 7 shows a close-up view of the edge profile thereof. In this embodiment, the strap 50 includes a center portion 52 that is flat (planar) and a pair of edge portions 54,55 that curl away from the skin-facing side 58 of the strap 50 as a comfort feature, so that the edges of the strap 50 are prevented from digging into the patient's skin when the strap 50 is wrapped around the patient's anatomy. The strap 50 further comprises an axial centerline 56.

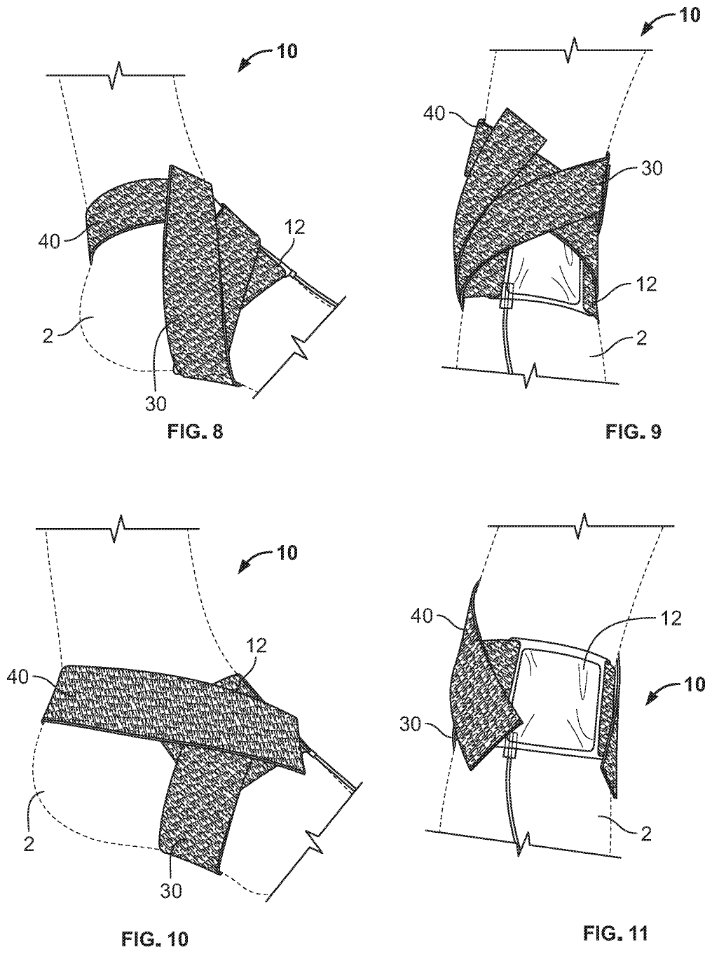

FIGS. 8-18 show various wrapping configurations and methods employing the hemostasis device 10 of FIG. 1 which incorporate the main portion 12 and the straps 30,40. It should be understood that any of these wrapping configurations and methods could utilize any of the embodiments of a hemostasis device taught herein, without departing from the spirit and scope of the present disclosure. It should be understood that the use of hemostasis devices on non-radial peripheral access points is not yet common, and that the anatomical structure of patients come in a wide variety of sizes and shapes. Further, the more uneven the anatomy, the greater the need for customizable fits.

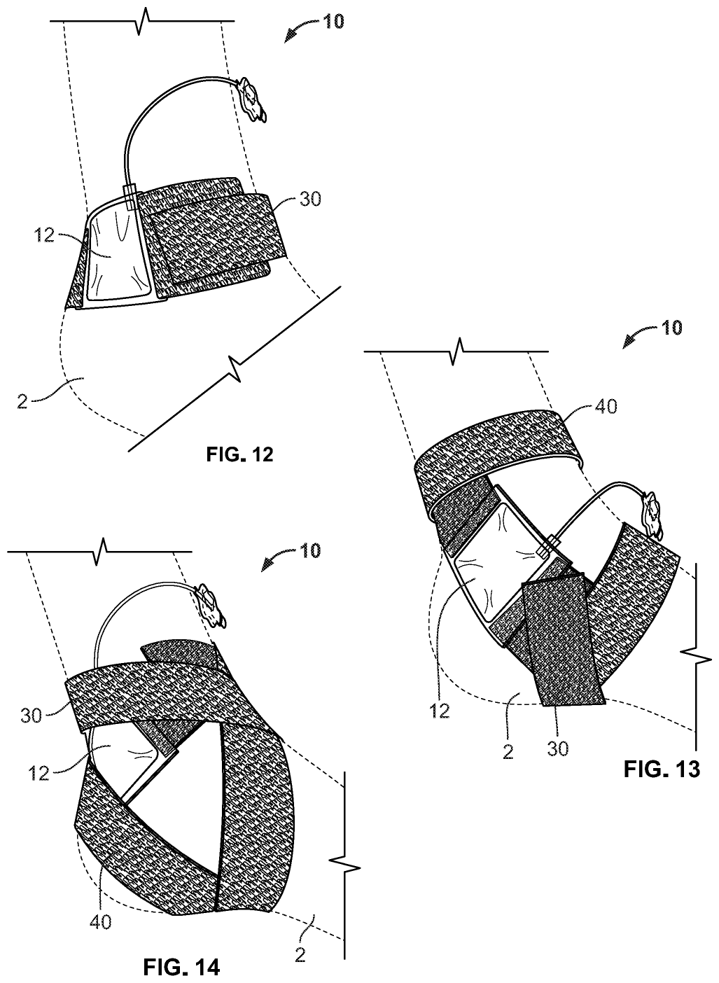

On one patient's foot, the best attachment angle for the hemostasis device may include having one strap wrapped around the ankle and another wrapped around the arch. On another patient's foot, a “Figure-8” shaped wrapping may provide a better fit. FIGS. 8 and 9 show the hemostasis device 10 of FIG. 1 attached to a foot 2 with the main portion 12 thereof atop a Dorsalis Pedis arterial access site, the first strap 30 wrapped around the arch of the foot 2, and the second strap 40 wrapped around the ankle of the foot 2, according to an embodiment of a wrapping configuration and method. FIGS. 10 and 11 show the hemostasis device 10 of FIG. 1 attached to a foot 2 with the main portion 12 thereof atop a Dorsalis Pedis arterial access site, the first strap 30 wrapped around the arch of the foot 2, and the second strap 40 wrapped around the ankle of the foot 2 according to another embodiment of a wrapping configuration and method.

FIG. 12 shows the hemostasis device 10 of FIG. 1 attached to a foot 2 with the main portion 12 thereof atop a Posterior Tibial arterial access site, the first strap 30 wrapped around the ankle of the foot 2, and with the second strap 40 not in use, according to another embodiment of a wrapping configuration and method. In this embodiment, the second strap 40 may not be necessary to hold the main portion 12 of the hemostasis device 10 properly in place on the ankle, given the generally-narrowing tapered shape of an ankle as it approaches the foot 2 and the presence of the large ankle bones to prevent migration of the main portion 12 past those bones.

FIG. 13 shows the hemostasis device 10 of FIG. 1 attached to a foot 2 atop a Posterior Tibial arterial access site, the first strap 30 wrapped around the arch of the foot 2, and the second strap 40 wrapped around the ankle of the foot, according to another embodiment of a wrapping configuration and method. FIG. 14 shows the hemostasis device 10 of FIG. 1 attached to a foot 2 atop a Posterior Tibial arterial access site, the first strap 30 wrapped around the ankle and arch of the foot, and the second strap 40 wrapped around the heel of the foot 2, according to yet another embodiment of a wrapping configuration and method.

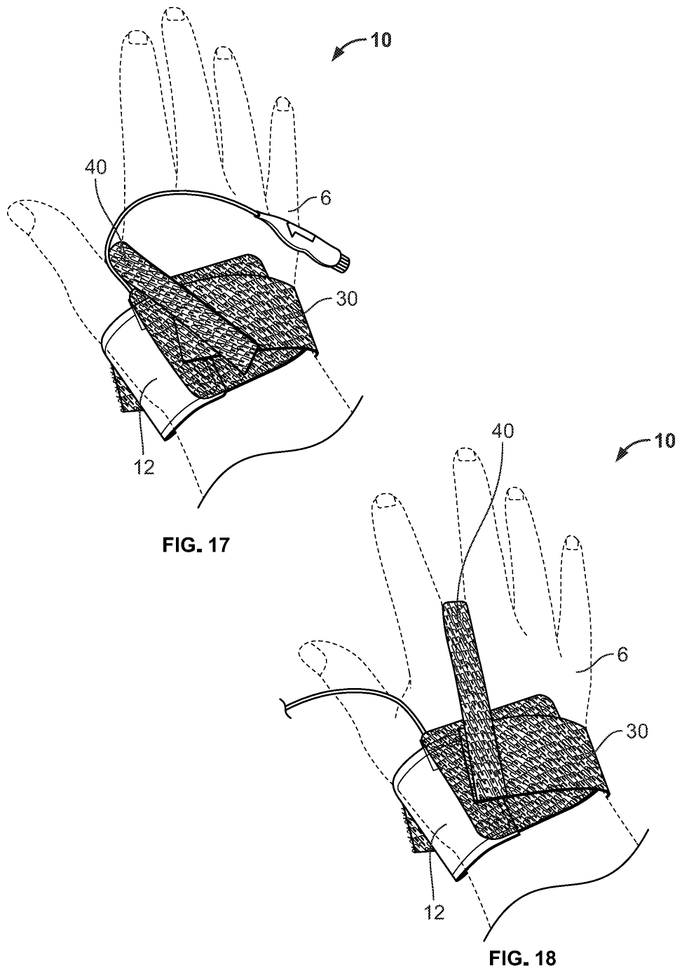

For distal radial (dTRA) arterial access, a placing a second strap between fingers or between the thumb and forefinger will help keep the balloon in place on the “snuff-box” area of the hand. A hemostasis device having adjustable strap locations and angles may be preferable to a device having fixed strap sizes, angles, and locations because various factors would result in the need for many pre-sized products just for hemostasis following dTRA procedures, due to various factors including variations in hand size, different placement for left- and right-hand use, and the need to make adjustments to avoid discomfort to the webbing of the patient's hand. FIGS. 15 and 16 show the main portion 12 of the hemostasis device 10 of FIG. 1 attached atop a Distal Radial arterial access site on a left hand 4, according to an embodiment of a wrapping configuration and method, in which the first strap 30 is wrapped around the wrist of the hand 4 and the second strap 40 is placed between the thumb and index finger of the hand 4. FIG. 17 shows a main portion 12 of the hemostasis device 10 of FIG. 1 attached atop a Distal Radial arterial access site on a right hand 6, according to another embodiment of a wrapping configuration and method, in which the first strap 30 is wrapped around the wrist of the hand 6 and the second strap 40 is placed between the thumb and index finger of the hand 6. FIG. 18 shows a main portion 12 of the hemostasis device 10 of FIG. 1 attached atop a Distal Radial arterial access site on a right hand 6, according to yet another embodiment of a wrapping configuration and method, in which the first strap 30 is wrapped around the wrist of the hand 6 and the second strap 40 is placed between the index and middle fingers of the hand 6. In the embodiments shown in FIGS. 15-18, the second strap 40 does not directly contribute to the application of hemostatic force to the access site, but is used primarily to keep the hemostasis device 10 in place. Moreover, as noted above, the second strap 40 in these embodiments could be elasticized for comfort.

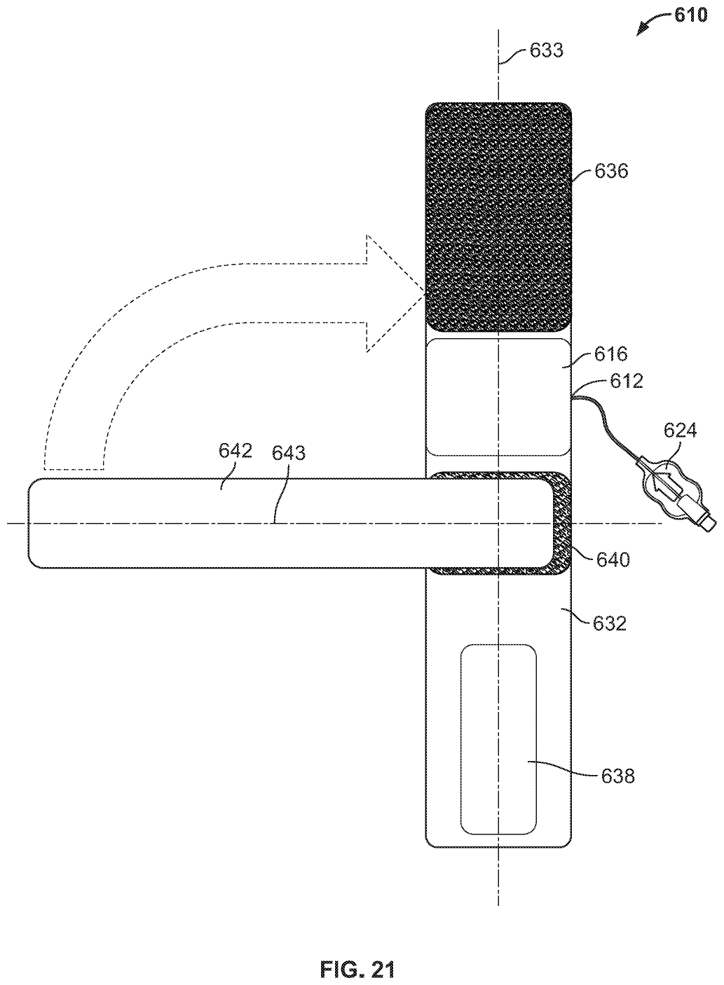

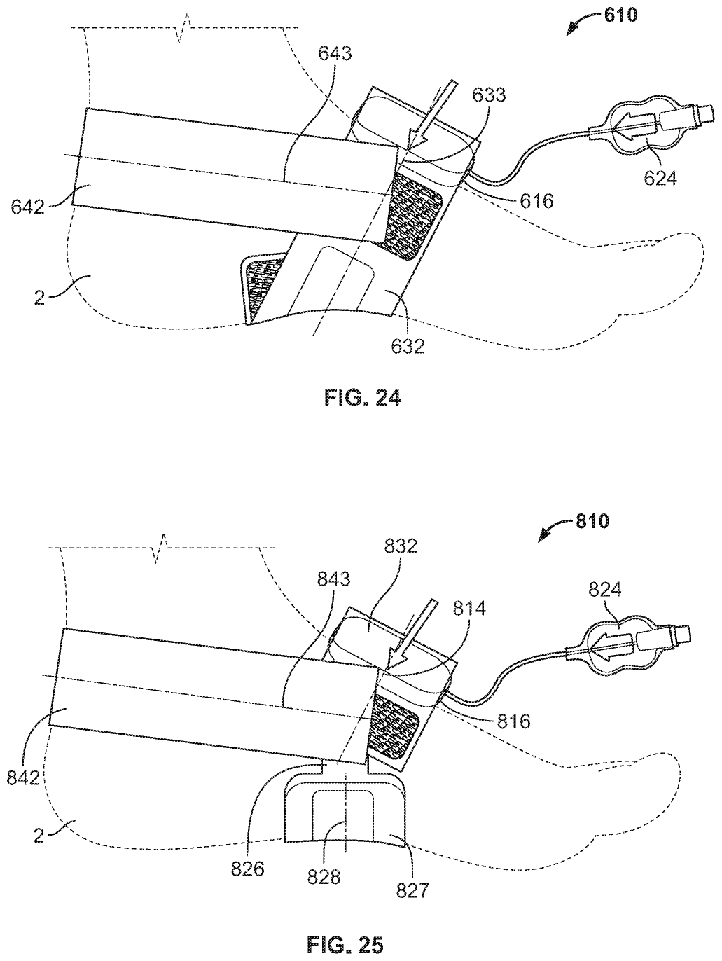

FIGS. 21-39 show various embodiments of hemostasis devices having customizable strap arrangements, according to the present disclosure. FIG. 21 shows an embodiment of a hemostasis device (band)610 which includes a main portion 612 having at least one balloon 616 that is inflatable via an inflator 624, a first strap 632 (which in this embodiment is integral with the main portion 612), and a second strap 642. The first strap 632/main portion 612 has an axial centerline 633 and the second strap 642 has an axial centerline 643. The first strap 632 further includes attachment portions 636,638 at ends thereof which are fastener halves that are attachable together to close the first strap 632 around a selected piece of anatomy while the balloon 616 is placed atop the access site. In addition, the first strap 632 includes an attachment portion 640 which is a fastener half that is attachable to the second strap 642. The other end of the second strap 642 is then attachable to the attachment portion 636 (see the arrow in FIG. 21) to close the second strap 642 around the patient's anatomy in a configuration in which its axial centerline 643 is not aligned with the axial centerline 633 of the first strap 632/main portion 612. FIG. 24 shows a wrapping configuration and method for the hemostasis device 610 of FIG. 21 on a foot 2, with the arrow showing a direction of the normal force applied by the balloon 616 to the access site.

In all of the embodiments discussed herein, the term “end” with respect to a strap can refer to either an actual end or to an “effective end” of the strap as supplied, with the “effective ends” of the strap collectively defining a circumference of the effective length of the strap once it has been wrapped and secured around a piece of anatomy. Said another way, in some embodiments according to the present disclosure, a fastener or fastener half (or multiple, separate fasteners or fastener halves)—or portions thereof—may be located away from an actual end of the strap as supplied, and an “effective end” of the strap may be defined by the location of the fastener or fastener half (or portion thereof) that is actually used by the clinician to secure the strap around the patient's anatomy, even if said fastener or fastener half (or utilized portion thereof) is located apart from the actual end of the strap. Where excess actual length portion(s) of a strap are present after the strap is wrapped and secured around a patient's anatomy to define the effective length of the strap, it should be understood that these excess actual length portion(s) may be trimmed off, tucked under the remainder of the strap, wrapped around the remainder of the strap, taped to the remainder of the strap, or left loose.

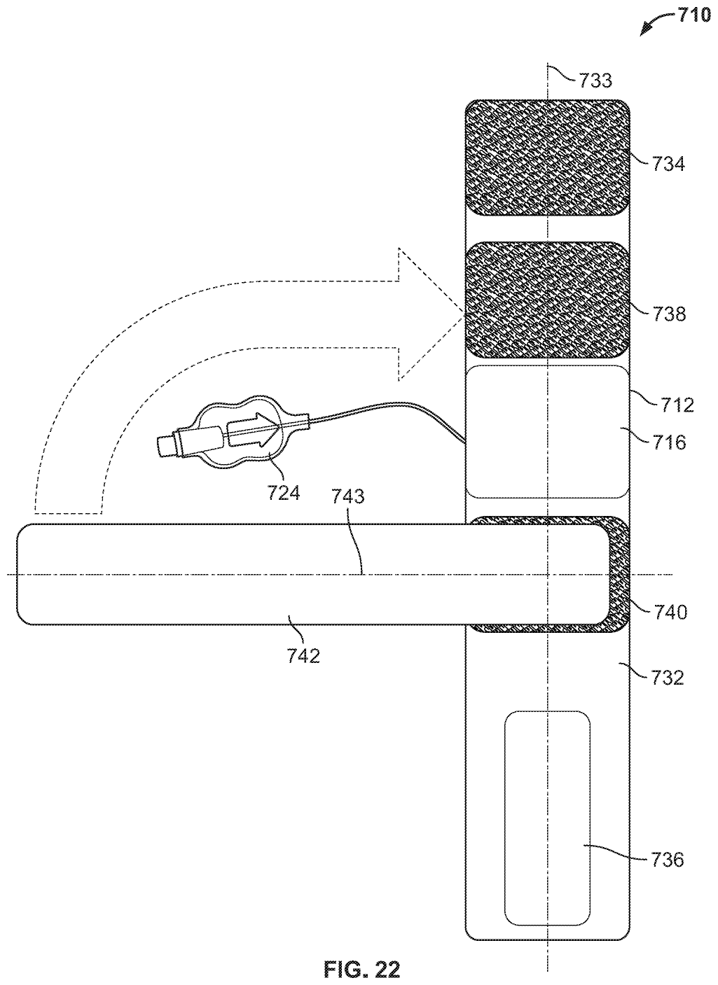

FIG. 22 shows another embodiment of a hemostasis device (band)710 which includes a main portion 712 having at least one balloon 716 that is inflatable via an inflator 724, a first strap 732 (which in this embodiment is integral with the main portion 712), and a second strap 742. The first strap 732/main portion 712 has an axial centerline 733 and the second strap 742 has an axial centerline 743. The first strap 732 further includes attachment portions 734,736 at ends thereof which are fastener halves that are attachable together to close the first strap 732 around a selected piece of anatomy while the balloon 716 is placed atop the access site. In addition, the first strap 732 includes an attachment portion 740 which is a fastener half that is attachable to one end of the second strap 742, and an attachment portion 738 which is a fastener half that is attachable to the other end of the second strap 742. In this embodiment, the second strap 742 is attachable between the attachment portions 740,738 (see the arrow in FIG. 22) to close the second strap 742 around the patient's anatomy in a configuration in which its axial centerline 743 is not aligned with the axial centerline 733 of the first strap 732/main portion 712.

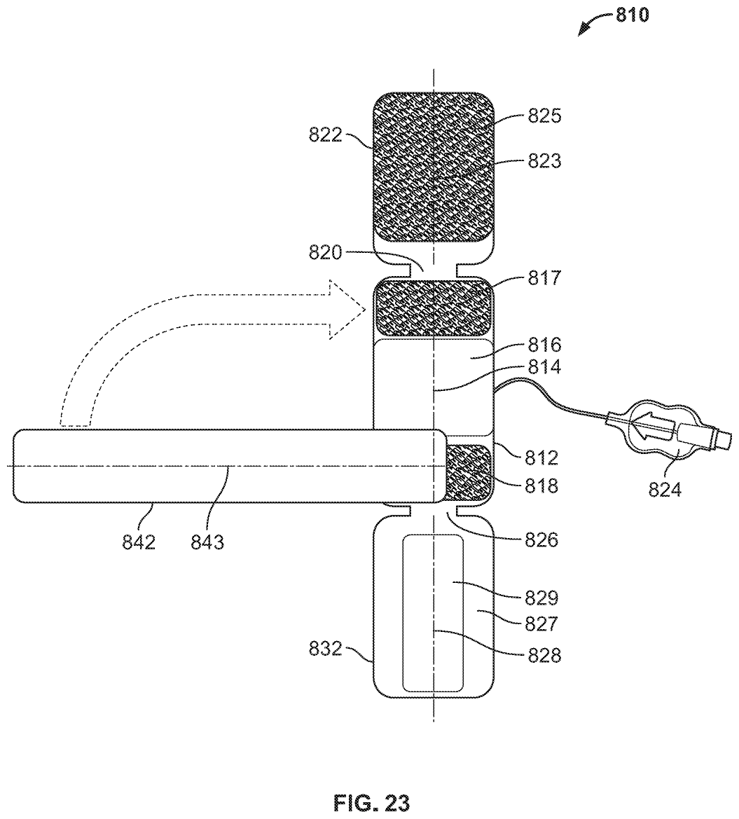

FIG. 23 shows another embodiment of a hemostasis device (band)810 which includes a main portion 812 having an axial centerline 814, at least one balloon 816 that is inflatable via an inflator 824, a first side portion 822 that is attached to the main portion 812 by a narrow portion 820, and a second side portion 827 that is attached to the main portion 812 by a narrow portion 826. The side portion 822 has an axial centerline 823 and an attachment portion 825 and the side portion 827 has an axial centerline 828 and an attachment portion 829. The attachment portions 825,829 are fastener halves that are attachable together to form a first strap 832 that is closeable around a selected piece of anatomy while the balloon 816 is placed atop the access site. The hemostasis device 810 further comprises a second strap 842 having an axial centerline 843. The main portion 812 further comprises attachment portions 817,818 which are fastener halves to which opposite ends of the second strap 842 are attachable (see the arrow in FIG. 23) to close the second strap 842 around the patient's anatomy. In this embodiment, the narrow portions 820,826 act as pivot locations that permit either or both of the axial centerlines 823,828 of the side portions 820,827 to be non-aligned with the axial centerline 814 of the main portion 812. Additionally, the axial centerline 843 of the second strap 842 can be placed in a non-aligned arrangement with respect to any or all of the axial centerlines 814,823,828 of the main portion 812 or side portions 820,827. FIG. 25 shows a wrapping configuration and method for the hemostasis device 810 of FIG. 23 on a foot 2, with the arrow showing a direction of the normal force applied by the balloon 816 to the access site.

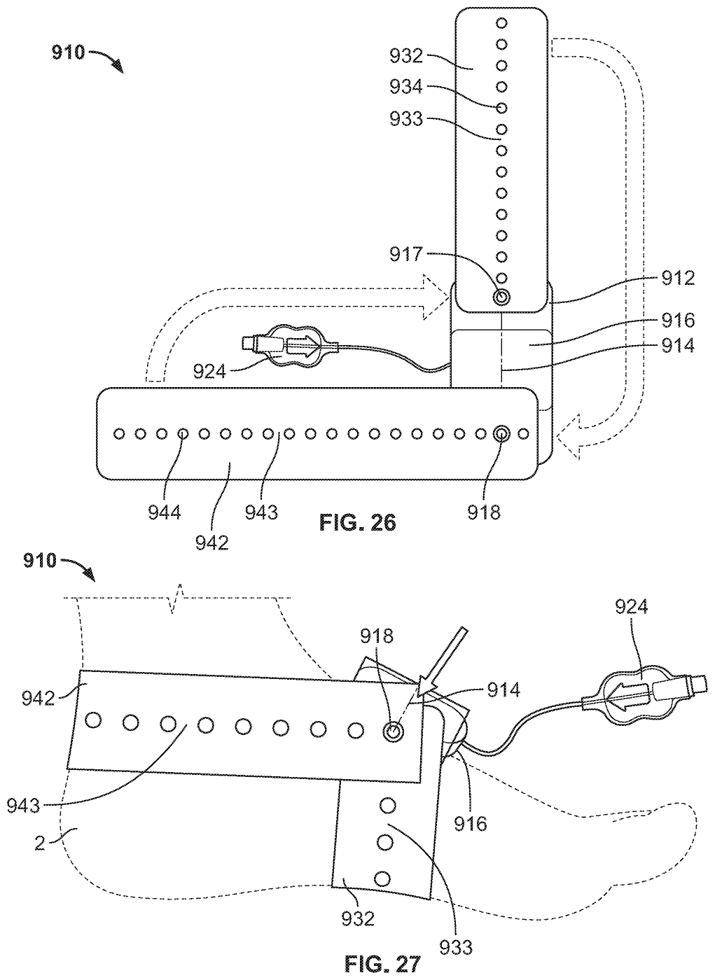

FIG. 26 shows another embodiment of a hemostasis device (band)910 which includes a main portion 912 having an axial centerline 914, at least one balloon 916 that is inflatable via an inflator 924, a first strap 932, and a second strap 942. In this embodiment, the main portion 912 comprises a rigid plate that includes two attachment portions 917,918 in the form of posts, prongs, brads, or buttons. In this embodiment, the first strap 932 includes a plurality of attachment portions in the form of holes (only one hole 934 labeled) aligned along an axial centerline 933 of the first strap 932, and the second strap 942 includes a plurality of attachment portions in the form of holes (only one hole 944 labeled) aligned along an axial centerline 943 of the second strap 942. The attachment portions 917,918 (i.e., posts) located on the main portion 912 are each adapted to be releasably connectable to any of the attachment portions (including holes 934,944) of each of the straps 932,942, so that each of the straps 932,942 can be attached at a first end thereof to a respective one of the attachment portions 917,918 (i.e., posts) located on the main portion 912, wrapped around a selected piece of anatomy, and then attached at a respective second end thereof to the other of the attachment portions 917,918 (i.e., posts) located on the main portion 912 (see arrows in FIG. 26). The plurality of attachment portions (including holes 934,944) located on each of the straps 932,942 allows for the effective length of each strap 932,942 to be customized by the clinician according to the anatomy to which the hemostasis device 910 is being applied. Further, either or both ends of both straps 932,942 are pivotable about the attachment portions 917,918 (i.e., posts) located on the main portion 912, which permits the axial centerlines 933,943 of either or both straps 932,942 to be placed in a non-aligned relationship with respect to the axial centerline 914 of the main portion 912. FIG. 27 shows a wrapping configuration and method for the hemostasis device 910 of FIG. 26 on a foot 2, with the arrow showing a direction of the normal force applied by the balloon 916 to the access site.

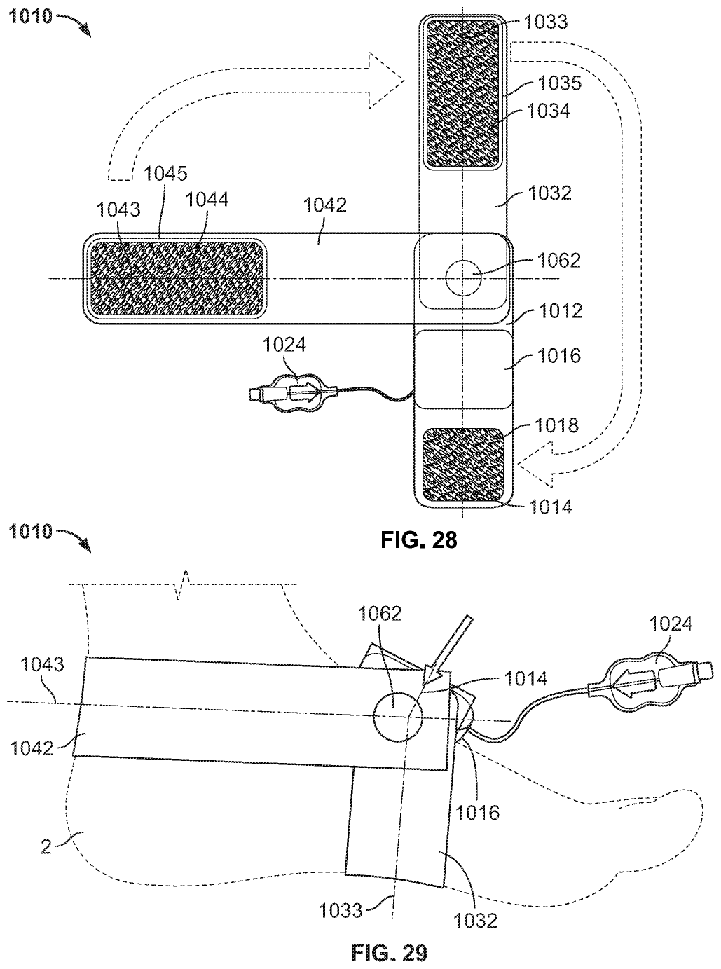

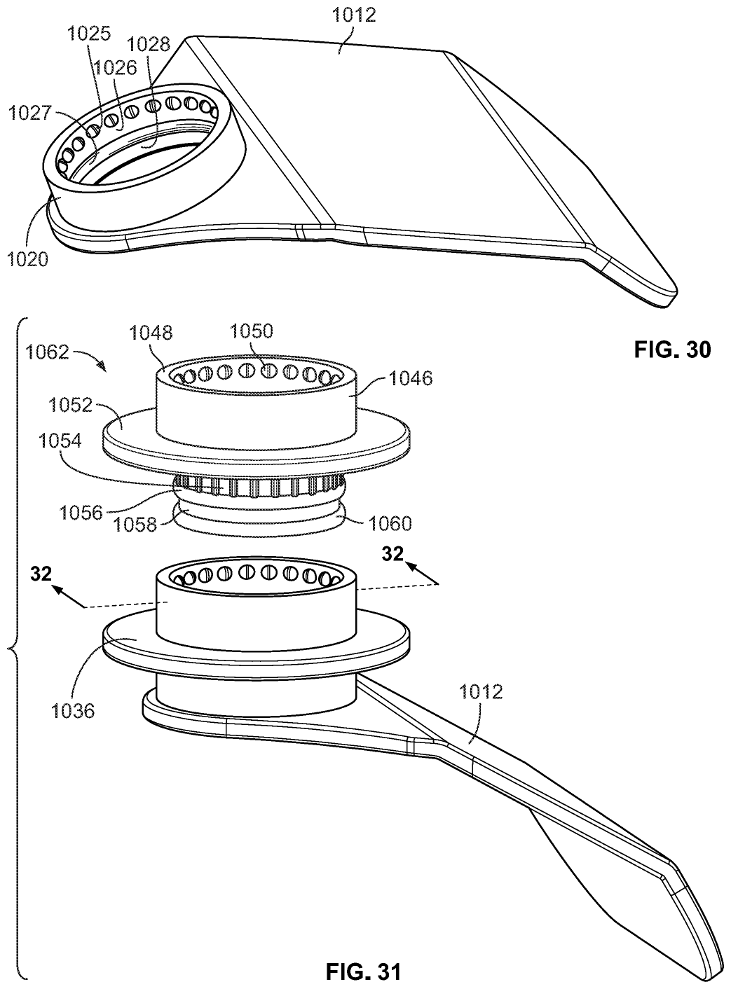

FIG. 28 shows another embodiment of a hemostasis device (band) 1010 which includes a main portion 1012 having an axial centerline 1014, at least one balloon 1016 that is inflatable via an inflator 1024, a first strap 1032, and a second strap 1042. In this embodiment, the main portion 1012 comprises a rigid plate that includes an attachment portion 1018 which is a fastener half, and an attachment portion 1020 (see FIGS. 30-32) of a rotating assembly 1062 by which the main portion 1012 is rotatably attachable to the first strap 1032 and the second strap 1042, as will be described below in further detail. The first strap 1032 has an axial centerline 1033 and the second strap 1042 has an axial centerline 1043. The first strap 1032 further includes a first attachment portion 1034 and a second attachment portion 1035 (located underneath the first attachment portion 1034 on the opposing (i.e., interior) side of the first strap 1032) at one end thereof. The second strap 1042 further includes a first attachment portion 1044 and a second attachment portion 1045 (located underneath the first attachment portion 1044 on the opposing side (i.e., interior) of the second strap 1042) at one end thereof. The back-to-back placement of the attachment portions 1034,1035 and attachment portions 1044,1045 at ends of the respective straps 1032,1042 allows for the ends of the straps 1032,1042 to be layered and attached together, as further explained below. In this embodiment, the attachment portion 1035 of the first strap 1032 is attachable with the attachment portion 1018 located on the main portion 1012 (see arrow on right side in FIG. 28) to close the first strap 1032 around a selected piece of anatomy while the balloon 1016 is placed atop the access site. The attachment portion 1045 of the second strap 1042 is then attachable to the attachment portion 1034 of the first strap 1032, in a layered fashion, to close the second strap 1042 around a selected piece of anatomy (see arrow on left side in FIG. 28). The attachment portion 1044 may then be used to attached another strap (not shown) on top of the second strap 1042 in a layered fashion, or could be omitted from the second strap 1042 entirely if the hemostasis device 1010 is designed to include no more than two straps. The rotating assembly 1062 allows the axial centerlines 1014,1033,1043 of each of the main portion 1012, first strap 1032, and second strap 1042 to be placed in non-aligned relationships with respect to each other while the hemostasis device 1010 is in use. FIG. 29 shows a wrapping configuration and method for the hemostasis device 1010 of FIG. 28 on afoot 2, with the arrow showing a direction of the normal force applied by the balloon 1016 to the access site. It should be understood that while each of the second attachment portions 1035,1045 are shown slightly larger than the respective first attachment portions 1034,1044 in FIG. 28 for illustration purposes, each of the second attachment portions 1035,1045 could be larger, smaller, or the same size as the respective first attachment portion 1034,1044.

FIGS. 30-32 show details of the rotating assembly 1062 according to the present embodiment. FIG. 30 show a perspective view of the main portion 1012 (i.e., a rigid support plate) with the balloon 1016, inflator 1024, air tube (not labeled), and attachment portion 1018 omitted from view. In this embodiment the main portion 1012 comprises a mount portion 1020, which is the base element of the rotating assembly 1062. The mount portion 1020 includes a series of studs (only one stud 1025 labeled in the Figures for clarity) arranged around the inner perimeter within the mount portion 1020, with a slot 1026 located around the inner perimeter within the mount portion 1020 below the series of studs (including stud 1025), a protruding rail 1027 located around the inner perimeter within the mount portion 1020 below the slot 1026, and another slot 1028 located around the inner perimeter within the mount portion 1020 below the protruding rail 1027.

In this embodiment, the rotating assembly 1062 further includes rotating piece 1036 (which is fixedly attached to the first strap 1032) and rotating piece 1046 (which is fixedly attached to the second strap 1042). It should be understood that the rotating pieces 1036,1046 are identical, and that any number of rotating pieces could be used as part of the rotating assembly 1062 depending on the number of straps (or other attachment accessories) that are desired to be part of the hemostasis device 1010. Because the rotating pieces 1036,1046 are identical, it should be understood that any description contained herein that relates to one is equally applicable to the other.

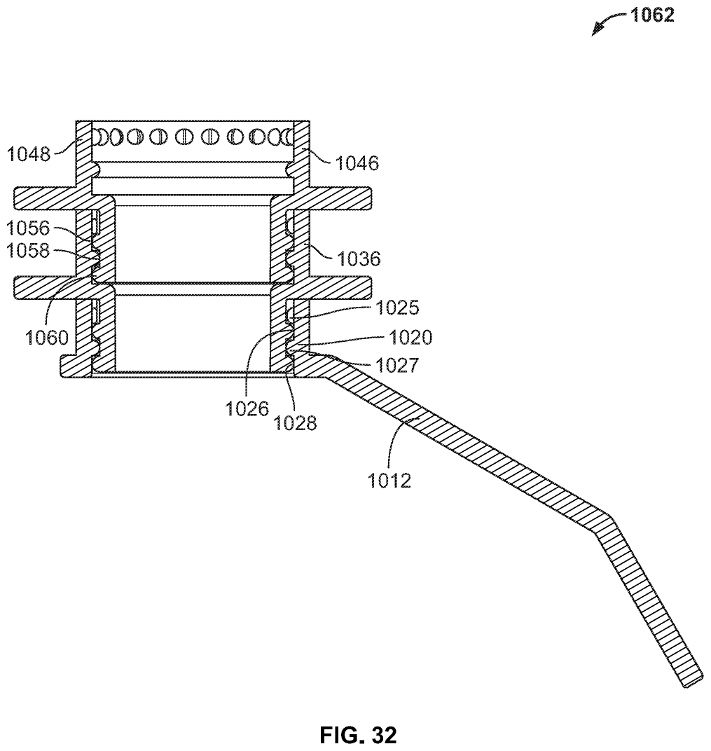

FIG. 31 shows a partial exploded view of the rotating assembly 1062 in which the rotating piece 1046 is shown separated from the rotating assembly 1062, and FIG. 32 shows a sectional view taken along line 32-32 of FIG. 31, showing a configuration of the rotating assembly 1062 in which the rotating piece 1046 is attached to the rotating piece 1036, with the rotating piece 1036 attached to the mount portion 1020. As shown in FIG. 31, the rotating piece 1046 comprises an upper portion 1048 above a lip portion 1052 thereof that is structurally identical to the structure of the mount portion 1020, i.e., comprising an array of studs (only one stud 1050 labeled in FIG. 31 for clarity) arranged around the inner perimeter within the upper portion 1048, with a slot located around the inner perimeter within the mount portion 1020 below the array of studs (including stud 1050), a protruding rail located around the inner perimeter within the mount portion 1020 below the slot, and another slot located around the inner perimeter within the mount portion 1020 below the protruding rail. Below the lip portion 1052, the rotating piece 1036 includes a series of detents (only one detent 1054 labeled in FIG. 31 for clarity) arranged around the outer perimeter, with a protruding rail 1056 located around the outer perimeter below the series of detents (including detent 1054), a slot 1058 located around the outer perimeter below the protruding rail 1056, and another protruding rail 1060 located around the outer perimeter below the slot 1058.

Because the parts of the upper rotating piece (i.e., rotating piece 1046) of the rotating assembly 1062 and the mount portion 1020 have been described above, we will discuss the connection between the rotating piece 1046 and mount portion 1020 below, though in the embodiment shown in FIGS. 31 and 32 rotating piece 1036 is connected between the rotating piece 1036 and the mount portion 1020. Since the rotating pieces 1036,1046 are identical, their stacking order in the rotating assembly 1062 could be swapped, or only one of the two rotating pieces 1036,1046 could be used in the rotating assembly 1062, it should be understood that the discussion below with respect to the connection between the mount portion 1020 and the rotating piece 1046 is equally applicable to a direct connection between the mount portion 1020 and the rotating piece 1036, and moreover that this discussion is equally applicable to a direct connection between any two rotating pieces according to the present embodiment.

In this embodiment, the rotating piece 1046 is attachable to the mount portion 1020 in one of two positions: a first position in which the rail 1060 of the rotating piece 1046 is located within the slot 1026 of the mount portion 1020 and the rail 1056 of the rotating piece 1046 is located above the series of studs (including stud 1025) of the mount portion 1020, such that the rotating piece 1046 (and its attached strap 1042) is freely rotatable with respect to the mount portion 1020, and a second position (as shown in FIG. 32) in which the rotating piece 1046 is pressed down further such that the rails 1056,1060 of the rotating piece 1046 are located, respectively, above and below the rail 1027 of the mount portion 1020 (with the rail 1027 located within the slot 1058 of the rotating piece 1046), and the series of detents (including detent 1054) of the rotating piece 1046 are engaged with the series of studs (including stud 1025) of the mount portion 1020. In this second position, the studs and detents interact to permit continued rotation of the rotating piece 1046 (and its attached strap 1042) with respect to the mount portion 1020 when intentionally rotated, but with resistance to prevent accidental reorientation and a tactile and, optionally, an audible “click” to indicate when the rotational position has been changed. In alternative embodiments, the ability for two separate connections between the rotating piece 1046 and the mount portion 1020 could be eliminated and the rotating assembly 1062 could be provided with only one type of connection (either free movement or resisted movement via stud/detent engagement), with the appropriate changes made to the structure—as would be understood by a person having ordinary skill in the art—to make the structure of the rotating assembly 1062 simpler and more compact.

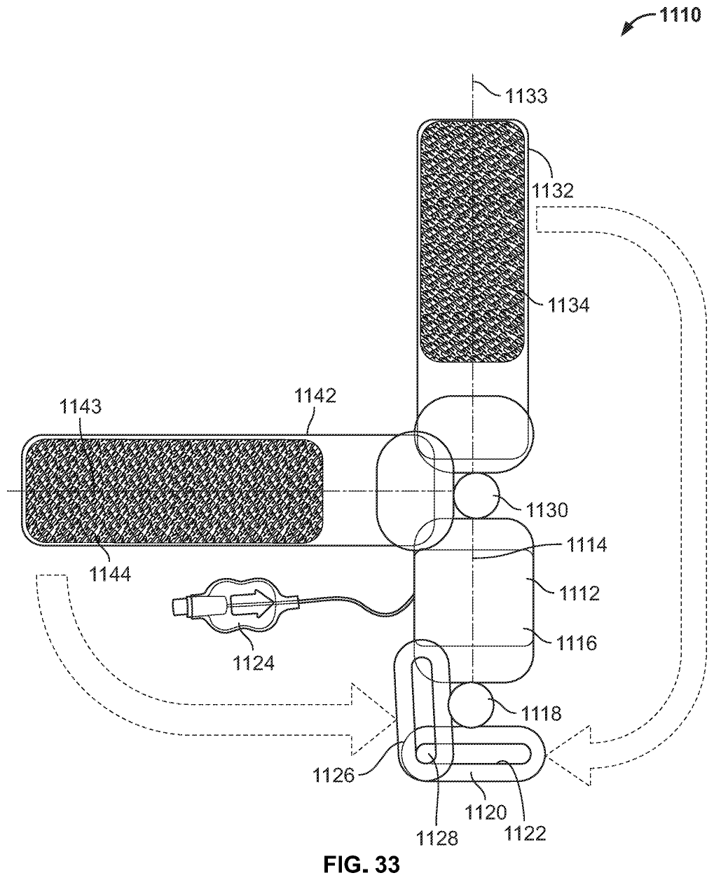

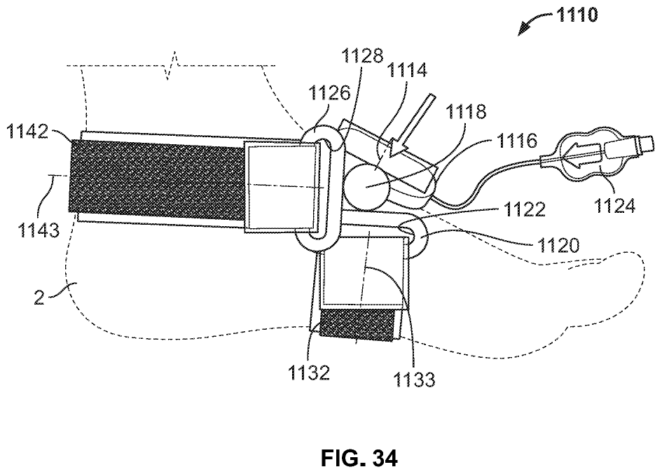

FIG. 33 shows another embodiment of a hemostasis device (band)1110 which includes a main portion 1112 having an axial centerline 1114, at least one balloon 1116 that is inflatable via an inflator 1124, a first strap 1132, and a second strap 1142. In this embodiment, the main portion 1112 comprises a rigid plate that includes two attachment portions 1118,1130 in the form of rotating assemblies, either or both of which could be of the structure of the rotating assembly 1062 as shown in FIGS. 28-32, or of any other type. The first strap 1132 has an axial centerline 1133 and includes an attachment portion 1134, and the second strap 1142 has an axial centerline 1143 and includes an attachment portion 1144. In this embodiment, each of the attachment portions 1134,1144 is in the form of a self-adhering fastener comprising both hook and loop material contained within the same fastener face, such that each of the attachment portions 1134,1144 is attachable to itself. In this embodiment, respective first ends of each of the first strap 1132 and second strap 1142 are rotatably attached to the main portion 1112 via the rotating assembly 1130. Loops 1120,1126 are each attached to the main portion 1112 via the other rotating assembly 1118, and each of the loops 1120,1126 is independently rotatable about the rotating assembly 1118. Each loop 1120,1126 includes a respective slot 1122,1128 therein through which an opposite end of a respective one of the straps 1132,1142 can be routed. In this embodiment, each of the first strap 1132 and second strap 1142 can be wrapped around a selected piece of anatomy, routed through a respective one of the loops 1120,1126 (see arrows in FIG. 33) to a desired extent to select an effective length of each strap 1132,1142, and then folded back and attached to itself to close the respective strap 1132,1142 around the selected piece of anatomy while the balloon 1116 is placed atop the access site. The rotating assemblies 1118,1130 allow the axial centerlines 1114,1133,1143 of each of the main portion 1112, first strap 1132, and second strap 1142 to be placed in non-aligned relationships with respect to each other while the hemostasis device 1110 is in use. FIG. 34 shows a wrapping configuration and method for the hemostasis device 1110 of FIG. 33 on a foot 2, with the arrow showing a direction of the normal force applied by the balloon 1116 to the access site.

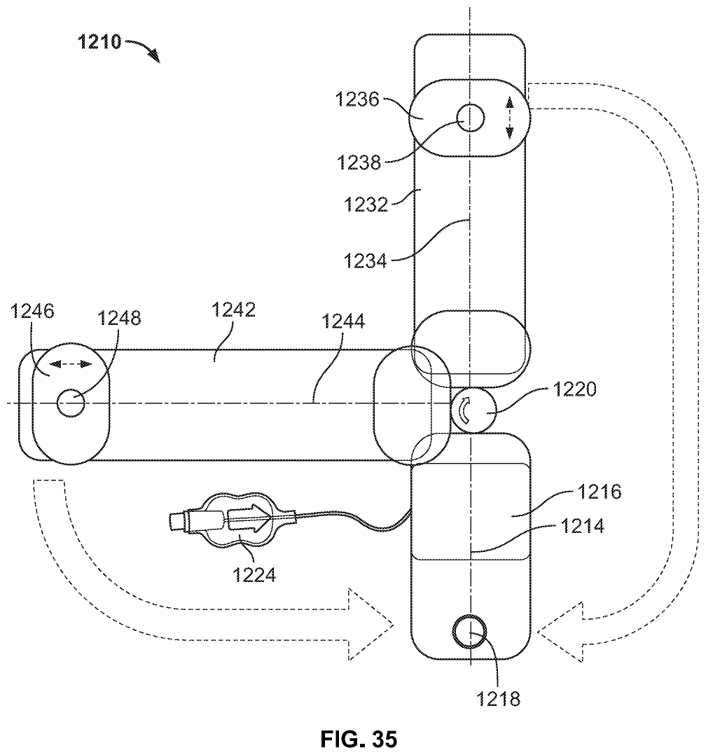

FIG. 35 shows another embodiment of a hemostasis device(band) 1210 which includes a main portion 1212 having an axial centerline 1214, at least one balloon 1216 that is inflatable via an inflator 1224, a first strap 1232, and a second strap 1242. In this embodiment, the main portion 1212 comprises a rigid plate that includes an attachment portion 1218 in the form of a post and an attachment portion 1220 in the form of a rotating assembly, which could be of the structure of the rotating assembly 1062 as shown in FIGS. 28-32, or of any other type. The first strap 1232 has an axial centerline 1234 and includes a clamp 1236 having an attachment portion 1238 in the form of a post located on a top side thereof and a bore (not shown) on the bottom side thereof that is sized and shaped to releasably engage with the attachment portion 1218 (i.e., post)located on the main portion 1212 (or with the attachment portion 1248 of the clamp 1246 of the second strap 1242). Similarly, the second strap 1242 has an axial centerline 1244 and includes a clamp 1246 having an attachment portion 1248 in the form of a post located on a top side thereof and a bore (not shown) on the bottom side thereof that is sized and shaped to releasably engage with the attachment portion 1218 (i.e., post) located on the main portion 1212 (or with the attachment portion 1238 of the clamp 1236 of the first strap 1232). In this embodiment, the position of each of the clamps 1236,1246 is adjustable along the length of the respective strap 1232,1242 (i.e., in directions parallel to the respective axial centerline 1234,1244 thereof), which allows for an effective length of each of the first strap 1232 and second strap 1242 to be adjusted. In this embodiment, respective first ends of each of the first strap 1232 and second strap 1242 are rotatably attached to the main portion 1212 via the rotating assembly 1220. Each of the first strap 1232 and second strap 1242 can be wrapped around a selected piece of anatomy and then rotatably attached to the attachment portion 1218 (i.e., post) of the main portion 1212 (either directly or indirectly via the respective attachment portion 1238,1248 of the other one of the clamps 1236,1246; see arrows in FIG. 35) while the balloon 1216 is placed atop the access site, to close the straps 1232,1242. The rotating assembly 1220 and the rotatable engagement of the clamps 1236,1246 with each other and the attachment portion 1218 (i.e., post) allow the axial centerlines 1214,1234,1244 of each of the main portion 1212, first strap 1232, and second strap 1242 to be placed in non-aligned relationships with respect to each other while the hemostasis device 1210 is in use.

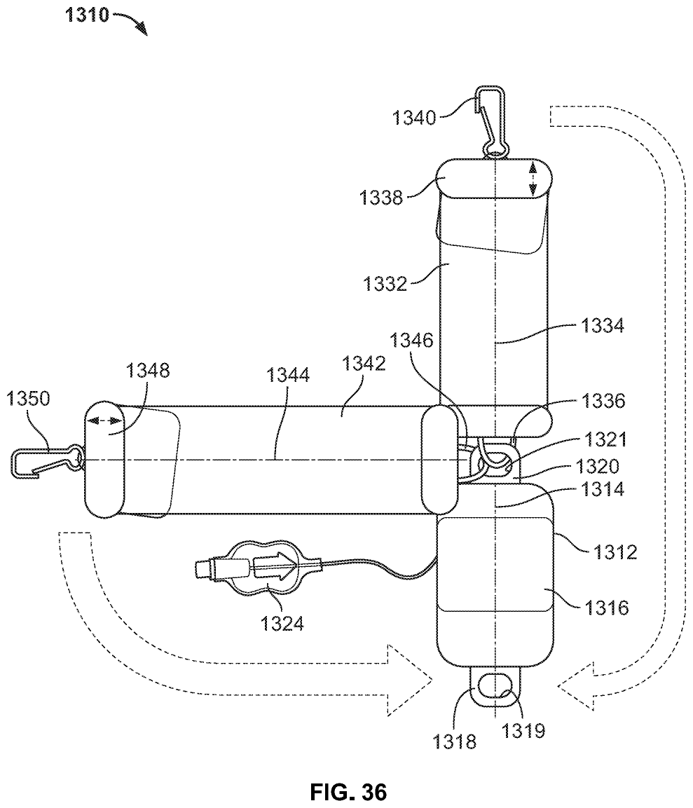

FIG. 36 shows another embodiment of a hemostasis device(band) 1310 which includes a main portion 1312 having an axial centerline 1314, at least one balloon 1316 that is inflatable via an inflator 1324, a first strap 1332, and a second strap 1342. In this embodiment, the main portion 1312 comprises a rigid plate that includes two attachment portions 1318,1320 in the form of tabs that extend from the main portion 1312 and each form a respective loop 1319,1321. The first strap 1332 has an axial centerline 1334 and an attachment portion 1336 in the form of a loop that is irremovably but rotatably attached to the loop 1321 of the main portion 1312, and further includes a clamp 1338 having an attachment portion 1340 in the form of a clip attached thereto that is adapted to be releasably attached to the attachment portion 1318 located on the main portion 1312. Similarly, the second strap 1342 has an axial centerline 1344 and an attachment portion 1346 in the form of a loop that is irremovably but rotatably attached to the loop 1321 of the main portion 1312, and further includes a clamp 1348 having an attachment portion 1350 in the form of a clip attached thereto that is adapted to be releasably attached to the attachment portion 1318 located on the main portion 1312. In this embodiment, the position of each of the clamps 1338,1348 is adjustable along the length of the respective strap 1332,1342 (i.e., in directions parallel to the respective axial centerline 1334,1344 thereof), which allows for an effective length of each of the first strap 1332 and second strap 1342 to be adjusted. Each of the first strap 1332 and second strap 1342 can be wrapped around a selected piece of anatomy and then rotatably attached to the attachment portion 1318 (i.e., tab with loop 1319) of the main portion 1312 while the balloon 1316 is placed atop the access site, to close the straps 1332,1342. The pivotable connections between both ends of each of the straps 1332,1342 and the two attachments portions 1318,1320 located on the main portion 1312 allow the axial centerlines 1314,1334,1344 of each of the main portion 1312, first strap 1332, and second strap 1342 to be placed in non-aligned relationships with respect to each other while the hemostasis device 1310 is in use. In alternative embodiments, one or both of the loops 1336,1346 could be replaced with a clip (e.g., the same type of clip used for attachment portions 1340,1350), so that either or both of the first strap 1332 and second strap 1342 are detachable from the main portion 1312.

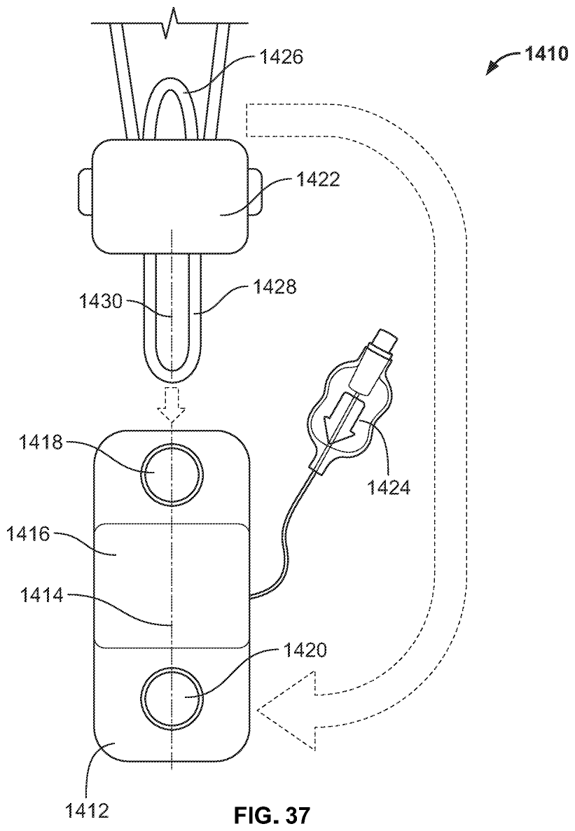

FIG. 37 shows another embodiment of a hemostasis device (band) 1410 which includes a main portion 1412 having an axial centerline 1414, at least one balloon 1416 that is inflatable via an inflator 1424, and one or more cord cinchers 1422 of known type to act as the first strap and any additional straps. In this embodiment, the main portion 1412 comprises a rigid plate that includes two attachment portions 1418,1420 in the form of posts, prongs, brads, or buttons that extend from the main portion 1412. The cord cincher 1422 includes a first attachment portion 1426 in the form of a loop that is attachable to the attachment portion 1420, and a second attachment portion 1428 in the form of an adjustable-sized loop (via the known adjustability of a cord cincher) that is attachable to the attachment portion 1418. The loop 1428 constitutes the first strap and includes an axial centerline 1430. A second cord cincher (not shown) could be separately attached between the attachment portions 1418,1420 on the main portion 1412 to constitute a second strap of the hemostasis device 1410. The cord cincher(s) each allow for an effective length of each of the first strap 1428 and any second strap to be adjusted, as would be understood of one having ordinary skill in the art. Each of the first strap 1428 and optional second strap can be wrapped around a selected piece of anatomy and attached at opposite ends thereof between the attachment portions 1418,1420 on the main portion 1412 (see arrows in FIG. 37). Since each of the connections is by a loop, the connections are pivotable such that the axial centerlines 1414,1430 of each of the main portion 1412, first strap 1428, and any optional second strap, can be placed in non-aligned relationships with respect to each other while the hemostasis device 1410 is in use.

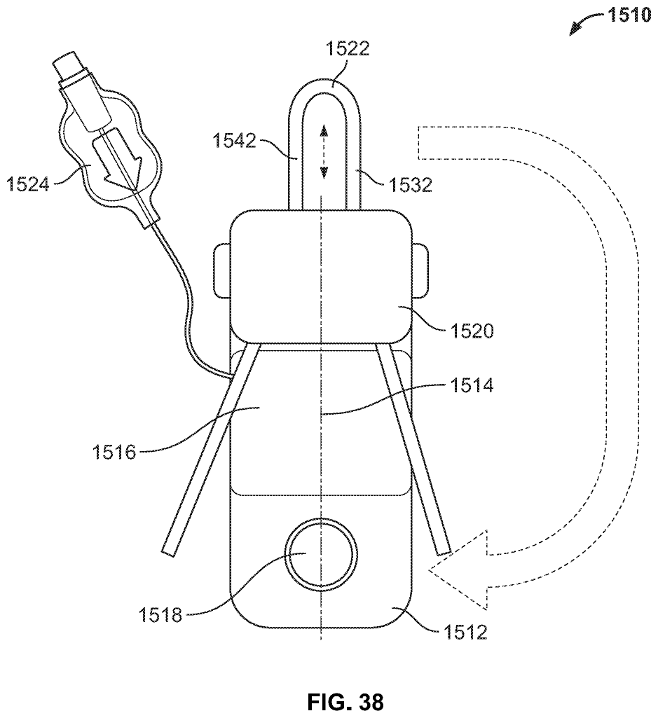

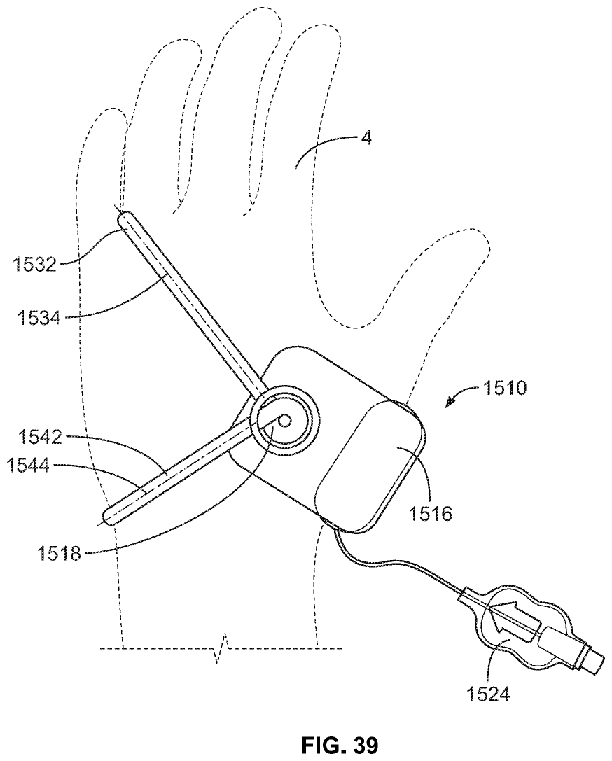

FIG. 38 shows another embodiment of a hemostasis device (band)1510 which includes a main portion 1512 having an axial centerline 1514, at least one balloon 1516 that is inflatable via an inflator 1524, and a built-in or fixedly-attached cord cincher 1520 of known type. In this embodiment, the main portion 1512 comprises a rigid plate that includes an attachment portion 1518 in the form of a post, prong, brad, or button that extends from the main portion 1512. The cord cincher 1520 includes an attachment portion 1522 in the form of an adjustable-sized loop (via the known adjustability of a cord cincher) that is attachable to the attachment portion 1518 on the main portion 1512. In this embodiment, the loop 1522 could be placed around a body part (e.g., a thumb or finger) such that the two halves of the cord routed to and from the cord cincher 1520 (i.e., the length of cord that form the loop 1522) constitute, respectively, the first strap 1532 and second strap 1542 during certain configurations and methods of use of the hemostasis device 1510. Such a wrapping configuration and method of use of the hemostasis device 1510 according to the present embodiment is shown in FIG. 39, in which the hemostasis device 1510 is attached to a hand 4 with the balloon 1516 located above a dTRA arterial access site in the snuff-box area of the hand 4. In this embodiment, the portion of the loop 1522 that constitutes the first strap 1532 has an axial centerline 1534 and the portion of the loop 1522 that constitutes the second strap 1542 has an axial centerline 1544, and the routing of the cord between the fingers of the hand 4 places the axial centerlines of the first strap 1532 and second strap 1542 in non-aligned relationships with respect to each other and with respect to the axial centerline of the main portion 1512, while the hemostasis device 1510 is in use. In an alternative embodiment according to FIGS. 38 and 39, two entirely separate pieces of cord could be separately attached to the cord cincher 1520 and the main portion 1512, with each of the separate pieces of cord comprising one of the two straps.

While the principles of the claimed invention have been described above in connection with specific embodiment(s), it is to be clearly understood that this description is made only by way of example and not as a limitation of the scope of the invention, as set forth in the appended claims.

1. A hemostasis device comprising:
a main portion, the main portion comprising at least one compression element that is adapted to be placed atop a site on a patient where bleeding is to be stopped, and at least one attachment portion, the at least one compression element capable of generating an applied force;
a first strap, the first strap being adapted to be wrapped around a body part of the patient and releasably attached to itself or to the at least one attachment portion of the main portion, the first strap having an axial centerline, the first strap being resistant to stretching such that it directs the applied force of the at least one compression element toward the site on the patient when the first strap is wrapped and releasably attached around the body part with the at least one compression element located atop the site on the patient; and
a second strap, the second strap being adapted to be wrapped around a body part of the patient and releasably attached to the main portion or the first strap, the second strap having an axial centerline,
wherein the second strap may be oriented at at least two different angles such that its axial centerline is not aligned with the axial centerline of the first strap in either of the at least two different angles.

2. The hemostasis device of claim 1, wherein the at least one compression element comprises at least one inflatable balloon, the at least one inflatable balloon comprising a first layer that is adjacent to the skin of the patient when the hemostasis device is being employed and a second layer opposite the first layer, wherein the second layer is less compliant to stretching than the first layer as the at least one inflatable balloon is inflated.

3. The hemostasis device of claim 1, the main portion further comprising a rigid plate or semi-rigid layer that is comprised of a material that is less elastic than the material from which the first strap is comprised.

4. The hemostasis device of claim 3, wherein the at least one compression element comprises at least one inflatable balloon, and wherein the at least one inflatable balloon is attached to the rigid plate or semi-rigid layer.

5. The hemostasis device of claim 3, the at least one rigid plate or semi-rigid layer including the at least one attachment portion.

6. The hemostasis device of claim 1, the main portion further comprising an axial centerline, wherein both of the first strap and the second strap may be oriented such that their respective axial centerlines are not aligned with the axial centerline of the main portion.

7. The hemostasis device of claim 1, wherein the second strap is comprised of a material that is more elastic than the material from which the first strap is comprised.

8. The hemostasis device of claim 1, wherein the main portion is integral with the first strap, the first strap further comprising at least one pivotable portion that separates the main portion from a side portion of the first strap, the main portion having an axial centerline and the side portion having an axial centerline, wherein the at least one pivotable portion permits the axial centerline of the side portion to be oriented such that is not aligned with the axial centerline of the main portion.

9. The hemostasis device of claim 8, wherein the at least one pivotable portion is comprised of a portion of material having a width that is less than the widths of each of the main portion and the side portion adjacent to the at least one pivotable portion.

10. The hemostasis device of claim 1, wherein both of the first strap and the second strap have effective lengths which are variable.

11. The hemostasis device of claim 10, wherein at least one of the first strap and the second strap has a movable element that varies the effective length thereof.

12. The hemostasis device of claim 1, wherein either of the first strap and the second strap are entirely detachable from the main portion.

13. The hemostasis device of claim 1, wherein both of the first strap and the second strap are entirely detachable from the main portion.

14. A kit for promoting hemostasis at a site on a patient, the kit comprising:
a main portion, the main portion comprising at least one compression element and an axial centerline;
a first strap, the first strap being removably attachable at both ends thereof to the main portion, the first strap further comprising an axial centerline; and
a second strap, the second strap being removably attachable at both ends thereof to either the main portion or the first strap, the second strap further comprising an axial centerline;
wherein each of said axial centerlines may be arranged in a non-linear configuration with respect to each other when the kit is being employed on the patient for the promotion of hemostasis at the site.

15. The kit of claim 14, the main portion further comprising a rigid plate or semi-rigid layer that is comprised of a material that is less elastic than the material from which the first strap is comprised.

16. The kit of claim 14, wherein the at least one compression element comprises at least one inflatable element.

17. The kit of claim 16, wherein the at least one compression element comprises a plurality of inflatable elements.

18. The kit of claim 17, wherein the plurality of inflatable elements comprises at least two inflatable balloons that press into each other when inflated to assist in the promotion of hemostasis at the site.

19. A kit for promoting hemostasis at a site on a patient, the kit comprising:
a main portion, the main portion comprising at least one compression element and a means for securing a plurality of straps thereto;
a first strap of the plurality of straps, the first strap being removably attachable at both ends thereof to the main portion via the means for securing; and
a second strap of the plurality of straps, the second strap being removably attachable at both ends thereof to the main portion via the means for securing.

20. The kit of claim 19, wherein the main portion comprises an axial centerline, the first strap comprises an axial centerline, and the second strap comprises an axial centerline, and each of said axial centerlines may be arranged in a non-linear configuration with respect to each other when the kit is being employed on a patient for the promotion of hemostasis.
